(19) United States
(12) Patent Application Publication
(10) Pub. No.: US 2011/0294836 A1
(43) Pub. Date: Dec. 1, 2011

(54) ARYL GPR119 AGONISTS AND USES THEREOF

(75) Inventors: Jiangao Song, Hayward, CA (US); Jingyuan Ma, Hayward, CA (US); Christopher J. Rabbat, Hayward, CA (US); Imad Nashashibi, Hayward, CA (US); Xin Chen, Hayward, CA (US); Zuchun Zhao, Hayward, CA (US)

(73) Assignee: METABOLEX, INC., Hayward, CA (US)

(21) Appl. No.: 13/000,868

(22) PCT Filed: Jun. 16, 2009
(86) PCT No.: PCT/US2009/047551

§ 371 Date: Jun. 3, 2011

Related U.S. Application Data
(60) Provisional application No. 61/074,552, filed on Jun. 20, 2008.

(51) Int. Cl. A61K 31/513; C07D 401/14; C07D 211/32; C07D 413/14; A61P 3/06; A61K 31/506; A61K 31/451; A61P 3/10; A61P 3/00; C07D 401/04; A61K 31/4545

(52) U.S. Cl. 514/269; 544/319; 546/194; 544/331; 544/333; 544/296; 546/226; 514/318; 514/275; 514/256; 514/330

(57) ABSTRACT

Aryl GPR119 agonists are provided. These compounds are useful for the treatment of diabetic diseases, including Type II diabetes and other diseases associated with poor glycemic control.

### BACKGROUND OF THE INVENTION

Diabetes mellitus can be divided into two clinical syndromes, Type I and Type II diabetes mellitus. Type I diabetes, or insulin-dependent diabetes mellitus, is a chronic autoimmune disease characterized by the extensive loss of beta cells in the pancreatic islets of Langerhans (hereinafter referred to as “pancreatic islet cells” or “islet cells”), which produce insulin. As these cells are progressively destroyed, the amount of secreted insulin decreases, eventually leading to hyperglycemia (abnormally high level of glucose in the blood) when the amount secreted drops below the level required for euglycemia (normal blood glucose level). Although the exact trigger for this immune response is not known, patients with Type I diabetes have high levels of antibodies against pancreatic beta cells (hereinafter “beta cells”). However, not all patients with high levels of these antibodies develop Type I diabetes.

Type II diabetes, or non-insulin-dependent diabetes mellitus, develops when muscle, fat and liver cells fail to respond normally to insulin. This failure to respond (called insulin resistance) may be due to reduced numbers of insulin receptors on these cells, or a dysfunction of signaling pathways within the cells, or both. The beta cells initially compensate for this insulin resistance by increasing their insulin output. Over time, these cells become unable to produce enough insulin to maintain normal glucose levels, indicating progression to Type II diabetes (Kahn S E, Am. J. Med. (2000) 108 Suppl 6a, 2S-8S).

The fasting hyperglycemia that characterizes Type II diabetes occurs as a consequence of the combined lesions of insulin resistance and beta cell dysfunction. The beta cell defect has two components: the first component, an elevation of basal insulin release (occurring in the presence of low, non-stimulatory glucose concentrations), is observed in obese, insulin-resistant pre-diabetic stages as well as in Type II diabetes. The second component is a failure to increase insulin release above the already elevated basal output in response to a hyperglycemic challenge. This lesion is absent in pre-diabetes and appears to define the transition from normo-glycemic insulin-resistant states to frank diabetes. There is currently no cure for diabetes. Conventional treatments for diabetes are very limited, and focus on attempting to control blood glucose levels in order to minimize or delay complications. Current treatments target either insulin resistance (metformin, thiazolidinediones (“TZDs”)), or insulin release from the beta cell (sulphonylureas, exenatide). Sulphonylureas, and other compounds that act by depolarizing the beta cell, have the side effect of hypoglycemia since they cause insulin secretion independent of circulating glucose levels. One approved drug, Byetta® (exenatide) stimulates insulin secretion only in the presence of high glucose, but is not orally available and must be injected. Januvia™ (sitagliptin) is another recently approved drug that increases blood levels of incretin hormones, which can increase insulin secretion, reduce glucagon secretion and have other less well characterized effects. However, Januvia™ and other dipeptidyl peptidases IV (DPP4) inhibitors may also influence the tissue levels of other hormones and peptides, and the long-term consequences of this broader effect have not been fully investigated. There is an unmet need for oral drugs that stimulate insulin secretion in a glucose dependent manner.

Progressive insulin resistance and loss of insulin secreting pancreatic beta cells are primary characteristics of Type II diabetes. Normally, a decline in the insulin sensitivity of muscle and fat is compensated for by increases in insulin secretion from the beta cell. However, loss of beta cell function and mass results in insulin insufficiency and diabetes (Kahn B B, Cell 92:593-596, 1998; Cavaghan M K, et al., J. Clin. Invest. 106:329-333, 2000; Saltiel A R, Cell 104:517-529, 2001; Prentki M and Nolan C J, J. Clin. Invest. 116:1802-1812 (2006); and Kahn S E, J. Clin. Endocrinol. Metab. 86:4047-4058, 2001). Hyperglycemia further accelerates the decline in beta cell function (UKPDS Group, J.A.M.A. 281:2005-2012, 1999; Levy J, et al., Diabetes Med. 15:290-296, 1998; and Zhou Y P, et al., J. Biol. Chem. 278:51316-23, 2003). Several of the genes in which allelic variation is associated with an increased risk of Type II diabetes are expressed selectively in the beta cell (Bell G I and Polonsky K S, Nature 414:788-791 (2001); Saxena R, et al., Science (2007) Apr. 26; [Epub ahead of print]; and Valgerdur Steinthorsdottir, et al., Nature Genetics (2007) Apr. 26; [Epub ahead of print]).

Insulin secretion from the beta cells of pancreatic islets is elicited by increased levels of blood glucose. Glucose is taken up into the beta cell primarily by the beta cell and liver selective transporter GLUT2 (Thorens B, Mol. Membr. Biol. 2001 October-December; 18(4):265-73). Once inside the cell, glucose is phosphorylated by glucokinase, which is the primary glucose sensor in the beta cell since it catalyzes the irreversible rate limiting step for glucose metabolism (Matschinsky F M, Curr. Diab. Rep. 2005 June; 5(3):171-6). The rate of glucose-6-phosphate production by glucokinase is dependent on the concentration of glucose around the beta cell, and therefore this enzyme allows for a direct relationship between level of glucose in the blood and the overall rate of glucose oxidation by the cell. Mutations in glucokinase produce abnormalities in glucose dependent insulin secretion in humans giving further evidence that this hexokinase family member plays a key role in the islet response to glucose (Gloyn A L, et al., J. Biol. Chem. 2005 Apr. 8; 280(14):14105-13. Epub 2005 Jan. 25). Small molecule activators of glucokinase enhance insulin secretion and may provide a route for therapeutic exploitation of the role of this enzyme (Guertin K R and Grimsby J, Curr. Med. Chem. 2006; 13(15):1839-43; and Matschinsky F M, et al., Diabetes 2006 January; 55(1):1-12) in diabetes. Glucose metabolism via glycolysis and mitochondrial oxidative phosphorylation ultimately results in ATP production, and the amount of ATP produced in a beta cell is directly related to the concentration of glucose to which the beta cell is exposed.

Elevated ratios of ATP to ADP that occur in the presence of higher glucose result in the closure of the Kir6.2 channel via interaction with the SUR1 subunit of the channel complex. Closure of these channels on the plasma membrane of the beta cell results in de-polarization of the membrane and subsequent activation of voltage dependent calcium channels (VDCCs) (Ashcroft F M and Dribble F M, Diabetologia 42:903-919, 1999; and Seino S, Annu. Rev. Physiol. 61:337-362, 1999). Calcium ion entry as well as release of calcium from intracellular stores triggers exocytosis of insulin granules, resulting in secretion of insulin into the blood stream. Agents which close the Kir6.2 channel such as sulphonylureas and metaglitinides (Rendell M, Drugs 2004; 64(12):1339-58; and Blickle J F, Diabetes Metab. 2006 April; 32(2):113-20) also cause membrane depolarization, and therefore these agents stimulate insulin secretion in a glucose independent fashion. Potassium channel openers, such as diazoxide, inhibit insulin secretion by preventing elevated ATP/ADP ratios from closing the Kir6.2 channel (Hansen J B, Curr. Med. Chem. 2006; 13(4):361-76). Calcium channel blockers, such as verapamil and nifedipine, can also inhibit insulin secretion (Henquin J C, (2004) Diabetes 53, S48-S58). Although sulfonylureas and metaglitinides are effective glucose lowering agents in the clinic, they act independently of blood glucose levels. Because they act independently of glucose levels, these drugs may result in hypoglycemia.

Glucose dependent insulin secretion from the beta cell is dependent on numerous neurotransmitters and blood-borne hormones, as well as local, intra-islet factors. CNS activation of the vagal innervation of the islet can lead to the release of small molecules such as acetylcholine and peptides such as vasoactive intestinal polypeptide (VIP), gastrin releasing peptide (GRP) and Pituitary Adenylate Cyclase Activating Peptide (PACAP). Acetylcholine activation of phospholipase C through the G_{αq}-coupled GPCR M3 muscarinic receptor leads to release of Ca++ from intracellular stores (Gilon P and Henquin J C, Endocr. Rev. 2001 October; 22(5):565-604). Cholinergic agonists also lead to a subtle Na+-dependent plasma membrane depolarization that can work in concert with glucose-initiated depolarization to enhance insulin release (Gilon P and Henquin J C, Endocr. Rev. 2001 October; 22(5):565-604). VIP and PACAP each bind to an overlapping set of G_{α}-coupled GPCRs (PAC1, VIPR1, and VIPR2) on the beta cell that lead to stimulation of adenylate cyclase and an increase in intracellular cAMP (Filipsson K, et al., Diabetes, 2001 September; 50(9):1959-69; Yamada H, et al., Regul. Pept. 2004 Dec. 15; 123(1-3):147-53; and Qader S S, et al., Am. J. Physiol. Endocrinol. Metab. 2007 May; 292(5):E1447-55).

Elevation of beta cell cAMP has a substantial potentiating effect on insulin secretion in the presence of stimulatory levels of glucose (see below). Unfortunately, many potentiators of glucose-stimulated insulin secretion also have effects outside of the islet which limit their ability to be used as diabetes therapeutics. For example, the best available selective muscarinic agonists which stimulate insulin secretion also stimulate multiple undesirable responses in multiple tissues (Rhoades R A and Tanner G A, eds. (2003) Medical Physiology, 2nd ed. Lippincott, Williams and Wilkins. ISBN 0-7817-1936-4). Likewise, VIP and PACAP receptors are present in multiple organ systems and mediate effects on the reproductive, immune and other diverse systems that make them less attractive as specific enhancers of glucose dependent insulin secretion.

Incretin hormones such as Glucagon-Like Peptide 1 (GLP-1) and Glucose-dependent Insulinotropic Polypeptide (GIP, also known as Gastric Inhibitory Polypeptide) also bind to specific Galphaₛ-coupled GPCRs receptors on the surface of islet cells, including beta cells, and raise intracellular cAMP (Drucker D J, J. Clin. Invest. 2007 January; 117(1):24-32). Although the receptors for these hormones are present in other cells and tissues, the overall sum of effects of these peptides appear to be beneficial to control of glucose metabolism in the organism (Hansotia T, et al., J. Clin. Invest. 2007 January; 117(1):143-52. Epub 2006 Dec. 21). GIP and GLP-1 are produced and secreted from intestinal K and L cells, respectively, and these peptide hormones are released in response to meals by both direct action of nutrients in the gut lumen and neural stimulation resulting from food ingestion. GIP and GLP-1 have short half-lives in human circulation due to the action of the protease dipeptidyl-peptidase IV (DPP4), and inhibitors of this protease can lower blood glucose due to their ability to raise the levels of active forms of the incretin peptides. The glucose lowering that can be obtained with DPP4 inhibitors, however, is somewhat limited since these drugs are dependent on the endogenous release of the incretin hormones. Peptides (e.g., exenatide (Byetta®)) and peptide-conjugates that bind to the GIP or GLP-1 receptors but are resistant to serum protease cleavage can also lower blood glucose substantially (Gonzalez C, et al., Expert Opin. Investig. Drugs 2006 August; 15(8):887-95), but these incretin mimetics must be injected and tend to induce a high rate of nausea and therefore are not ideal therapies for general use in the Type II diabetic population. The clinical success of DPP4 inhibitors and incretin mimetics, though far from ideal, do point to the potential utility of compounds that increase incretin activity in the blood or directly stimulate cAMP in the beta cell. Some studies have indicated that beta cell responsiveness to GIP is diminished in Type II diabetes (Nauck M A, et al., J. Clin. Invest. 91:301-307 (1993); and Elahi D, et al., Regul. Pept. 51:63-74 (1994)). Restoration of this responsiveness (Meneilly G S, et al., Diabetes Care. 1993 January; 16(1):110-4) may be a promising way to improve beta cell function in vivo.

Since increased incretin activity has a positive effect on glucose dependent insulin secretion and perhaps other mechanisms that lead to lower blood glucose, it is also of interest to explore therapeutic approaches to increasing incretin release from intestinal K and L cells. GLP-1 secretion appears to be attenuated in Type II diabetes (Vilsboll T, et al., Diabetes 50:609-613), so improving incretin release may ameliorate this component of metabolic dysregulation. Nutrients such as glucose and fat in the gut lumen prompt incretin secretion by interaction with apical receptors (Vilsboll T, et al., Diabetes 50:609-613). GLP-1 and GIP release can also result from neural stimulation; acetylcholine and GRP can enhance incretin release in a manner perhaps analogous to the effects of these neurotransmitters on the beta cell in regard to insulin secretion (Brubaker P, Ann. NY Acad. Sci. 2006 July; 1070:10-26; and Reimann F, et al., Diabetes 2006 December; 55 (Suppl 2):S78-S85). Somatostatin, leptin and free fatty acids also appear to modulate incretin secretion (Brubaker P, Ann. NY Acad. Sci. 2006 July; 1070:10-26; and Reimann F, et al., Diabetes 2006 December; 55(Suppl 2):S78-S85). To date, however, there does not appear to be a way to selectively impact these pathways to promote incretin secretion for therapeutic benefit. There is a need for oral drugs that stimulate incretin secretion in the treatment of diabetes.

Incretins can also increase the rate of beta cell proliferation and decrease the apoptotic rates of beta cells in animal models (Farilla L, et al., Endocrinology 2002 November; 143(11):4397-408) and human islets in vitro (Farilla L, et al., Endocrinology 2003 December; 144(12):5149-58). The net result of these changes is an increase in beta cell number and islet mass, and this should provide for increased insulin secretory capacity, which is another desired aim of anti-diabetic therapies. GLP-1 has also been shown to protect islets from the destructive effects of agents such as streptozotocin by blocking apoptosis (Li Y, et al., J. Biol. Chem. 2003 Jan. 3; 278(1):471-8). Cyclin D1, a key regulator of progression through the cell cycle, is up-regulated by GLP-1, and other agents that increase cAMP and PKA activity also have a similar effect (Friedrichsen B N, et al., J. Endocrinol. 2006 March; 188(3):481-92; and Kim M J, et al., J. Endocrinol. 2006 March; 188(3):623-33). Increased transcription of the cyclin D1 gene occurs in response to PKA phosphorylation of CREB (cAMP-response element binding) transcription factors (Hussain M A, et al., Mol. Cell. Biol. 2006 October; 26(20):7747-59). There is a need for oral drugs that increase beta cell number and islet mass in the treatment of diabetes.

Beta cell cAMP levels may also be raised by inhibiting the degradation of this second messenger by phosphodiesterases to AMP (Furman B and Pyne N, Curr. Opin. Investig. Drugs 2006 October; 7(10):898-905). There are several different cAMP phosphodiesterases in the beta cell, and many of these have been shown to serve as a brake on glucose-dependent insulin secretion Inhibitors of cAMP phosphodiesterases have been shown to increase insulin secretion in vitro and in vivo, including PDE1C, PDE3B, PDE10, (Han P, et al., J. Biol. Chem. 1999 Aug. 6; 274(32):22337-44; Harndahl L, et al., J. Biol. Chem. 2002 Oct. 4; 277(40):37446-55; Walz H A, et al., J. Endocrinol. 2006 June; 189(3):629-41; Choi Y H, et al., J. Clin. Invest. 2006 December; 116(12):3240-51; and Cantin L D, et al., Bioorg. Med. Chem. Lett. 2007 May 15; 17(10):2869-73) but so far, no PDEs have been found to have the cell type selectivity necessary to avoid undesirable effects. However, this remains an area of active investigation due to the potential for amplification of the effects of incretins and other agents that stimulate adenylate cyclase.

There appear to be multiple mechanisms by which cAMP elevation in the beta cell can enhance glucose dependent insulin secretion. Classically, many of the intracellular effects of cAMP are mediated by the cAMP-dependent protein kinase (protein kinase A, PKA) (Hatakeyama H, et al., J. Physiol. 2006 Jan. 15; 570(Pt 2):271-82). PKA consists of a complex of two regulatory and two catalytic domains; binding of cAMP to the catalytic domains releases the catalytic domains and results in increased protein phosphorylation activity. One of the downstream effects of this kinase activity is enhanced efficiency of insulin exocytosis (Gromada J, et al., Diabetes 1998 January; 47(1):57-65). Another cAMP binding protein is Epac, a guanine nucleotide exchange factor (GEF) (Kashima Y, et al., J. Biol. Chem. 2001 Dec. 7; 276(49):46046-53. Epub 2001 Oct. 11; and Shibasaki T, et al., J. Biol. Chem. 2004 Feb. 27; 279(9):7956-61), which mediates a cAMP-dependent, but PKA-independent, increase in insulin exocytosis. Epac activated by cAMP may also enhance of release of intracellular Ca++ (Holz G G, Diabetes 2004 January; 53(1):5-13). The effects of cAMP on insulin secretion are dependent on elevated glucose levels, so raising cAMP in the pancreatic beta cell is an important goal for therapeutics of Type II diabetes.

Agents that raise intracellular cAMP levels in the beta cell increase insulin secretion in a glucose dependent manner (MiuraY and Matsui H, Am. J. Physiol. Endocrinol. Metab. (2003) 285, E1001-E1009). One mechanism for raising cAMP is by the action of G-protein coupled cell surface receptors, which stimulate the enzyme adenylate cyclase to produce more cAMP. The GLP-1 receptor, which is the target of exenatide, is an example of such a receptor (Thorens B, et al., Diabetes (1993) 42, 1678-1682). There is a need for oral drugs that increase intracellular levels of cAMP in the treatment of diabetes.

DPP4 inhibitors are inhibitors of dipeptidyl peptidase-4. DPP4 is a prolyl protease that preferentially cleaves peptides after a proline amino acid residue. DPP4 is believed to degrade GLP-1. DPP4 inhibitors have been shown to prevent N-terminal degradation of GLP-1, and lowered blood glucose in preclinical studies. In addition, mice with a targeted disruption of the DPP4 gene had increased plasma levels of GLP-1 and GIP. Approved DPP4 inhibitors for treatment of diabetes include sitagliptin (Januvia™) and vildagliptin (Galvus™). Saxagliptin (BMS-477118) is another DPP4 inhibitor currently in clinical trials.

### BRIEF SUMMARY OF THE INVENTION

Novel GPR119 agonists are provided. The novel GPR119 agonists are useful in the treatment of diabetes and other related diseases including metabolic syndrome, dyslipidemia, insulin resistance, and complications of diabetes. GPR119 is also known as RUP3 and IC-GPCR2.

Agonists of GPR119 are also useful in raising intracellular cyclic adenosine monophosphate (cAMP) levels (see Biological Example 1). Such raised cAMP levels increase insulin secretion in a glucose dependent manner (see Biological Example 2) and thus provide a useful treatment for, inter alia, Type II diabetes. Biological Example 3 describes a widely practiced glucose tolerance test. Additionally, Biological Example 4 describes methods to determine the effect of GPR119 agonists on the secretion of incretins. Biological Example 5 shows methods of determining improvements in diabetes parameters widely accepted by skilled artisans in an animal diabetes model using ZDF rats. Agonists of GPR119 capable of raising intracellular cAMP levels have now been identified using a cell-based screen (see Biological Example 1).

The present invention provides compounds represented by Formula (I), (II) or (III) as shown below.

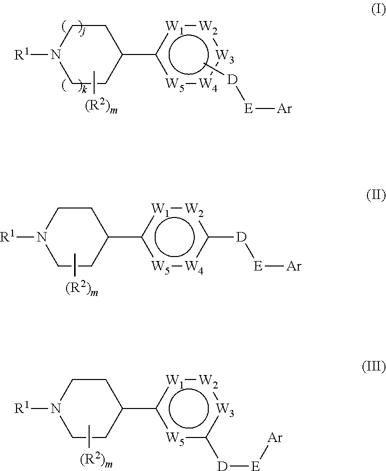

Also provided are pharmaceutically acceptable salts, solvates, stereoisomers and esters of the compounds of Formula (I), (II) or (III).

This invention also provides methods of treating diseases such as Type II diabetes and other diseases and conditions using one or more of these compounds or compositions, as described in further detail below. The invention also provides methods of raising intracellular levels of cyclic AMP (cAMP) by using one or more of the compounds described herein. Further, the compounds may be used to stimulate insulin production and stimulate secretion of insulin, glucagon-like peptide 1 (GLP1), and glucose dependent insulinotropic polypeptide (GIP) in a mammal, in particular a human. Additionally, the compounds described herein are useful in lowering blood glucose when administered to a subject in need of treatment to lower blood glucose.

In a related aspect, the present invention provides methods of diagnosing a number of diseases and conditions using labeled compounds of Formula (I), (II) or (III).

An aspect of this invention provides methods of lowering blood levels of glucose in a subject by administering to a patient in need thereof a compound of Formula (I), (II) or (III). This invention also provides methods of lowering blood levels of glucose in a subject by administering to a patient in need thereof a compound of Formula (I), (II) or (III) and a DPP4 inhibitor.

Another aspect of this invention provides methods of lowering blood levels of insulin in a subject by administering to a patient in need thereof a compound of Formula (I), (II) or (III). This invention further provides methods of lowering blood levels of insulin in a subject by administering to a patient in need thereof a compound of Formula (I), (II) or (III) and a DPP4 inhibitor.

In another aspect, this invention provides methods of increasing blood levels of incretins in a subject by administering to a patient in need thereof a compound of Formula (I), (II) or (III). Also provided are methods of increasing blood levels of incretins in a subject by administering to a patient in need thereof a compound of Formula (I), (II) or (III) and a DPP4 inhibitor. The incretins are GLP-1 and GIP.

Yet another aspect of this invention provides methods of lowering blood triglyceride levels in a patient by administering to a patient in need thereof a compound of Formula (I), (II) or (III). This invention provides methods of lowering blood triglyceride levels in a patient by administering to a patient in need thereof a compound of Formula (I), (II) or (III) and a DPP4 inhibitor.

A further aspect of this invention provides methods of lowering gastric emptying in a patient by administering to a patient in need thereof a compound of Formula (I), (II) or (III). Also provided are methods of lowering gastric emptying in a patient by administering to a patient in need thereof a compound of Formula (I), (II) or (III) and a DPP4 inhibitor.

Another aspect of this invention provides methods of increasing insulin production in the islet cells of a patient by administering to a patient in need thereof a compound of Formula (I), (II) or (III). Additionally, this invention provides methods of increasing insulin production in the islet cells of a patient by administering to a patient in need thereof a compound of Formula (I), (II) or (III) and a DPP4 inhibitor.

In yet another aspect, this invention provides methods of preserving islet function in a subject by administering to a patient in need thereof a compound of Formula (I), (II) or (III). In yet another aspect, this invention provides methods of preserving islet function in a subject by administering to a patient in need thereof a compound of Formula (I), (II) or (III) and a DPP4 inhibitor.

### DETAILED DESCRIPTION OF THE INVENTION

The abbreviations used herein are conventional, unless otherwise defined: AcOH: acetic acid; nBuLi: n-butyllithium; Cs₂CO₃: cesium carbonate; CH₂Cl₂ or DCM: dichloromethane; CH₃MgI: methyl magnesium iodide; CuCl₂: copper chloride; DAST: (diethylamino)sulfur trifluoride; DEAD: diethyl azodicarboxylate; DIBAL: diisobutylaluminum hydride; DIPEA: diisopropylethylamine; DMSO: dimethyl sulfoxide; Et₃N: triethylamine; EtOAc: ethyl acetate; H₂: hydrogen; HBr: hydrogen bromide; HCl: hydrogen chloride; H₂O: water; H₂O₂: hydrogen peroxide; HPLC: high performance liquid chromatography; KCN: potassium cyanide; LHMDS: lithium hexamethyldisilazide; LiAlH₄: lithium aluminum hydride; LiOH: lithium hydroxide; MeCN: acetonitrile; MeI: methyl iodide; MeOH: methanol; MgSO₄: magnesium sulfate; MgCO₃: magnesium carbonate; MsCl: mesyl chloride; NaHSO₃: sodium hydrogen sulfite; mCPBA: meta-chloroperoxybenzoic acid; N₂: nitrogen; Na₂CO₃: sodium carbonate; NaHCO₃: sodium bicarbonate; NaNO₂: sodium nitrite; NaOH: sodium hydroxide; Na₂S₂O₃: sodium bisulfate; Na₂SO₄: sodium sulfate; NBS:

N-bromosuccinimide; NH₄Cl: ammonium chloride; NH₄OAc: ammonium acetate; NMR: nuclear magnetic resonance; Pd/C: palladium on carbon; PPh₃: triphenyl phosphine; iPrOH: isopropyl alcohol; SOCl₂: thionyl chloride; THF: tetrahydrofuran; TLC: thin layer chromatography.

Unless otherwise stated, the following terms used in the specification and claims have the meanings given below:

“Alkyl” refers to monovalent saturated aliphatic hydrocarbyl groups having from 1 to 10 carbon atoms and, in some embodiments, from 1 to 6 carbon atoms. “Cᵤ₋ᵥalkyl” refers to alkyl groups having from u to v carbon atoms. This term includes, by way of example, linear and branched hydrocarbyl groups such as methyl(CH₃—), ethyl(CH₃CH₂—), n-propyl (CH₃CH₂CH₂—), isopropyl((CH₃)₂CH—), n-butyl(CH₃CH₂CH₂CH₂—), isobutyl ((CH₃)₂CHCH₂—), sec-butyl((CH₃)(CH₃CH₂)CH—), t-butyl((CH₃)₃C—), n-pentyl (CH₃CH₂CH₂CH₂CH₂—), and neopentyl((CH₃)₃CCH₂—).

“Substituted alkyl” refers to an alkyl group having from 1 to 5 and, in some embodiments, 1 to 3 or 1 to 2 substituents selected from the group consisting of alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, spirocycloalkyl, SO₃H, substituted sulfonyl, sulfonyloxy, thioacyl, thiocyanate, thiol, alkylthio, and substituted alkylthio, wherein said substituents are as defined herein.

“Alkylidene” or “alkylene” refers to divalent saturated aliphatic hydrocarbyl groups having from 1 to 10 carbon atoms and, in some embodiments, from 1 to 6 carbon atoms. “(Cᵤ₋ᵥ)alkylene” refers to alkylene groups having from u to v carbon atoms. The alkylidene and alkylene groups include branched and straight chain hydrocarbyl groups. For example “(C₁₋₆)alkylene” is meant to include methylene, ethylene, propylene, 2-methypropylene, pentylene, and the like.

“Substituted alkylidene” or “substituted alkylene” refers to an alkylidene group having from 1 to 5 and, in some embodiments, 1 to 3 or 1 to 2 substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, oxo, thione, spirocycloalkyl, SO₃H, substituted sulfonyl, sulfonyloxy, thioacyl, thiocyanate, thiol, alkylthio, and substituted alkylthio, wherein said substituents are as defined herein.

“Alkenyl” refers to a linear or branched hydrocarbyl group having from 2 to 10 carbon atoms and, in some embodiments, from 2 to 6 carbon atoms or 2 to 4 carbon atoms and having at least one site of vinyl unsaturation (>C═C<). For example, (Cᵤ₋ᵥ)alkenyl refers to alkenyl groups having from u to v carbon atoms and is meant to include for example, ethenyl, propenyl, 1,3-butadienyl, and the like.

“Substituted alkenyl” refers to alkenyl groups having from 1 to 3 substituents and, in some embodiments, 1 to 2 substituents, selected from the group consisting of alkoxy, substituted alkoxy, acyl, acylamino, acyloxy, alkyl, substituted alkyl, alkynyl, substituted alkynyl, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined as herein and with the proviso that any hydroxy or thiol substitution is not attached to an acetylenic carbon atom.

“Alkenylene” refers to divalent alkenyl groups having from 2 to 10 carbon atoms and, in some embodiments, from 2 to 6 carbon atoms. “(Cᵤ₋ᵥ)alkenylene” refers to alkenylene groups having from u to v carbon atoms.

“Alkynyl” refers to a linear monovalent hydrocarbon radical or a branched monovalent hydrocarbon radical containing at least one triple bond. The term “alkynyl” is also meant to include those hydrocarbyl groups having one triple bond and one double bond. For example, (C₂-C₆)alkynyl is meant to include ethynyl, propynyl, and the like.

“Substituted alkynyl” refers to alkynyl groups having from 1 to 3 substituents and, in some embodiments, from 1 to 2 substituents, selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiol, alkylthio, and substituted alkylthio, wherein said substituents are defined herein and with the proviso that any hydroxy or thiol substitution is not attached to an acetylenic carbon atom.

“Alkynylene” refers to divalent alkynyl groups having from 2 to 10 carbon atoms and, in some embodiments, from 2 to 6 carbon atoms. “(Cᵤ₋ᵥ)alkynylene” refers to alkynylene groups having from u to v carbon atoms.

“Alkoxy” refers to the group —O-alkyl wherein alkyl is defined herein. Alkoxy includes, by way of example, methoxy, ethoxy, n-propoxy, isopropoxy, n-butoxy, t-butoxy, sec-butoxy, and n-pentoxy.

“Substituted alkoxy” refers to the group —O-(substituted alkyl) wherein substituted alkyl is as defined herein.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, substituted hydrazino-C(O)—, heteroaryl-C(O)—, substituted heteroaryl-C(O)—, heterocyclic-C(O)—, and substituted heterocyclic-C(O)—, wherein alkyl, substituted alkyl, alkenyl, substituted alkenyl, alkynyl, substituted alkynyl, cycloalkyl, substituted cycloalkyl, aryl, substituted aryl, substituted hydrazino, heteroaryl, substituted heteroaryl, heterocyclic and substituted heterocyclic are as defined herein. Acyl includes the “acetyl” group CH₃C(O)—.

“Acylamino” refers to the groups —NR²⁰C(O)H, —NR²⁰C(O)alkyl, —NR²⁰C(O)substituted alkyl, —NR²⁰C(O)cycloalkyl, —NR²⁰C(O)substituted cycloalkyl, —NR²⁰C(O)alkenyl, —NR²⁰C(O)substituted alkenyl, —NR²⁰C(O)alkynyl, —NR²⁰C(O)substituted alkynyl, —NR²⁰C(O)aryl, —NR²⁰C(O)substituted aryl, —NR²⁰C(O)heteroaryl, —NR²⁰C(O)substituted heteroaryl, —NR²⁰C(O)heterocyclic, and —NR²⁰C(O)substituted heterocyclic wherein R²⁰ is hydrogen or alkyl and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Acyloxy” refers to the groups H—C(O)O—, alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O— wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“Amino” refers to the group —NH₂.

“Substituted amino” refers to the group —NR²¹R²² where R²¹ and R²² are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclyl, and —SO₂-substituted heterocyclyl and wherein R²¹ and R²² are optionally joined together with the nitrogen bound thereto to form a heterocyclyl or substituted heterocyclyl group, provided that R²¹ and R²² are both not hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein. When R²¹ is hydrogen and R²² is alkyl, the substituted amino group is sometimes referred to herein as alkylamino. When R²¹ and R²² are alkyl, the substituted amino group is sometimes referred to herein as dialkylamino. When referring to a monosubstituted amino, it is meant that either R²¹ or R²² is hydrogen but not both. When referring to a disubstituted amino, it is meant that neither R²¹ nor R²² are hydrogen.

“Hydroxyamino” refers to the group —NHOH.

“Alkoxyamino” refers to the group —NHO-alkyl wherein alkyl is defined herein.

“Aminocarbonyl” refers to the group —C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, hydroxy, alkoxy, substituted alkoxy, amino, substituted amino, and acylamino, and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonyl” refers to the group —C(S)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonylamino” refers to the group —NR²⁰C(O)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminothiocarbonylamino” refers to the group —NR²⁰C(S)NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminocarbonyloxy” refers to the group —O—C(O)NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyl” refers to the group —SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonyloxy” refers to the group —O—SO₂NR²³R²⁴ where R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aminosulfonylamino” refers to the group —NR²⁰—SO₂NR²³R²⁴ where R²⁰ is hydrogen or alkyl and R²³ and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Amidino” refers to the group —C(═NR²⁵)NR²³R²⁴ where R²⁵, R²³, and R²⁴ are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic and where R²³ and R²⁴ are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Aryl” refers to an aromatic group of from 6 to 14 carbon atoms and no ring heteroatoms and having a single ring (e.g., phenyl) or multiple condensed (fused) rings (e.g., naphthyl or anthryl). For multiple ring systems, including fused, bridged, and spiro ring systems having aromatic and non-aromatic rings that have no ring heteroatoms, the term “Aryl” or “Ar” applies when the point of attachment is at an aromatic carbon atom (e.g., 5,6,7,8-tetrahydronaphthalene-2-yl is an aryl group as its point of attachment is at the 2-position of the aromatic phenyl ring).

“Substituted aryl” refers to aryl groups which are substituted with 1 to 8 and, in some embodiments, 1 to 5, 1 to 3 or 1 to 2 substituents selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are defined herein.

“Arylalkyl” or “Aryl(C₁-C_{z})alkyl” refers to the radical —R^{u}R^{v} where R^{u} is an alkylene group (having 8 or fewer main chain carbon atoms) and R^{v} is an aryl group as defined herein. Thus, “arylalkyl” refers to groups such as, for example, benzyl, and phenylethyl, and the like. Similarly, “Arylalkenyl” means a radical —R^{u}R^{v} where R^{u} is an alkenylene group (an alkylene group having 1 or 2 double bonds) and R^{v} is an aryl group as defined herein, e.g., styrenyl, 3-phenyl-2-propenyl, and the like.

“Aryloxy” refers to the group —O-aryl, where aryl is as defined herein, that includes, by way of example, phenoxy and naphthoxy.

“Substituted aryloxy” refers to the group —O-(substituted aryl) where substituted aryl is as defined herein.

“Arylthio” refers to the group —S-aryl, where aryl is as defined herein.

“Substituted arylthio” refers to the group —S-(substituted aryl), where substituted aryl is as defined herein.

“Azido” refers to the group —N₃.

“Hydrazino” refers to the group —NHNH₂.

“Substituted hydrazino” refers to the group —NR²⁶NR²⁷R²⁸ where R²⁶, R²⁷, and R²⁸ are independently selected from the group consisting of hydrogen, alkyl, substituted alkyl, alkenyl, substituted alkenyl, alkynyl, substituted alkynyl, aryl, substituted aryl, carboxyl ester, cycloalkyl, substituted cycloalkyl, heteroaryl, substituted heteroaryl, heterocyclic, substituted heterocyclic, —SO₂-alkyl, —SO₂-substituted alkyl, —SO₂-alkenyl, —SO₂-substituted alkenyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, and —SO₂-substituted heterocyclic and wherein R²⁷ and R²⁸ are optionally joined, together with the nitrogen bound thereto to form a heterocyclic or substituted heterocyclic group, provided that R²⁷ and R²⁸ are both not hydrogen, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cyano” or “carbonitrile” refers to the group —CN.

“Carbonyl” refers to the divalent group —C(O)— which is equivalent to —C(═O)—.

“Carboxyl” or “carboxy” refers to —COOH or salts thereof.

“Carboxyl ester” or “carboxy ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)β-cycloalkyl, —C(O)O-substituted cycloalkyl, —C(O)O-heteroaryl, —C(O)O-substituted heteroaryl, —C(O)O-heterocyclic, and —C(O)O-substituted heterocyclic wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)amino” refers to the group —NR²⁰—C(O)O-alkyl, —NR²⁰—C(O)O-substituted alkyl, —NR²⁰—C(O)O-alkenyl, —NR²⁰—C(O)O-substituted alkenyl, —NR²⁰—C(O)O-alkynyl, —NR²⁰—C(O)O-substituted alkynyl, —NR²⁰—C(O)O-aryl, —NR²⁰—C(O)O-substituted aryl, —NR²⁰—C(O)O-cycloalkyl, —NR²⁰—C(O)O-substituted cycloalkyl, —NR²⁰—C(O)O-heteroaryl, —NR²⁰—C(O)O-substituted heteroaryl, —NR²⁰—C(O)O-heterocyclic, and —NR²⁰—C(O)O-substituted heterocyclic wherein R²⁰ is alkyl or hydrogen, and wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic, and substituted heterocyclic are as defined herein.

“(Carboxyl ester)oxy” refers to the group —O—C(O)O-alkyl, —O—C(O)O-substituted alkyl, —O—C(O)O-alkenyl, —O—C(O)O-substituted alkenyl, —O—C(O)O-alkynyl, —O—C(O)O-substituted alkynyl, —O—C(O)O-aryl, —O—C(O)O-substituted aryl, —O—C(O)O-cycloalkyl, —O—C(O)O-substituted cycloalkyl, —O—C(O)O-heteroaryl, —O—C(O)O-substituted heteroaryl, —O—C(O)O-heterocyclic, and —O—C(O)O-substituted heterocyclic wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Cycloalkyl” refers to a saturated or partially saturated cyclic group of from 3 to 14 carbon atoms and no ring heteroatoms and having a single ring or multiple rings including fused, bridged, and spiro ring systems. For multiple ring systems having aromatic and non-aromatic rings that have no ring heteroatoms, the term “cycloalkyl” applies when the point of attachment is at a non-aromatic carbon atom (e.g., 5,6,7,8,-tetrahydronaphthalene-5-yl). The term “cycloalkyl” includes cycloalkenyl groups. Examples of cycloalkyl groups include, for instance, adamantyl, cyclopropyl, cyclobutyl, cyclopentyl, cyclooctyl, and cyclohexenyl. “Cᵤ₋ᵥcycloalkyl” refers to cycloalkyl groups having u to v carbon atoms as ring members. “Cᵤ₋ᵥcycloalkenyl” refers to cycloalkenyl groups having u to v carbon atoms as ring members.

“Cycloalkenyl” refers to a partially saturated cycloalkyl ring having at least one site of >C═C< ring unsaturation.

“Substituted cycloalkyl” refers to a cycloalkyl group, as defined herein, having from 1 to 8, or 1 to 5, or, in some embodiments, 1 to 3 substituents selected from the group consisting of oxo, thione, alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, azido, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, guanidino, substituted guanidino, halo, hydroxy, hydroxyamino, alkoxyamino, hydrazino, substituted hydrazino, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, substituted sulfonyl, sulfonyloxy, thioacyl, thiocyanate, thiol, alkylthio, and substituted alkylthio, wherein said substituents are as defined herein. The term “substituted cycloalkyl” includes substituted cycloalkenyl groups.

“Cycloalkyloxy” refers to —O-cycloalkyl wherein cycloalkyl is as defined herein.

“Substituted cycloalkyloxy” refers to —O-(substituted cycloalkyl) wherein substituted cycloalkyl is as defined herein.

“Cycloalkylthio” refers to —S-cycloalkyl wherein substituted cycloalkyl is as defined herein.

“Substituted cycloalkylthio” refers to —S-(substituted cycloalkyl) wherein substituted cycloalkyl is as defined herein.

“Guanidino” refers to the group —NHC(═NH)NH₂.

“Substituted guanidino” refers to —NR²⁹C(═NR²⁹)N(R²⁹)₂ where each R²⁹ is independently selected from the group consisting of hydrogen, alkyl, substituted alkyl, aryl, substituted aryl, heteroaryl, substituted heteroaryl, heterocyclyl, and substituted heterocyclyl and two R²⁹ groups attached to a common guanidino nitrogen atom are optionally joined together with the nitrogen bound thereto to form a heterocyclic or substituted heterocyclic group, provided that at least one R²⁹ is not hydrogen, and wherein said substituents are as defined herein.

“Halo” or “halogen” refers to fluoro, chloro, bromo and iodo.

“Haloalkyl” refers to substitution of alkyl groups with 1 to 5 or, in some embodiments, 1 to 3 halo groups, e.g., —CH₂Cl, —CH₂F, —CH₂Br, —CFClBr, —CH₂CH₂Cl, —CH₂CH₂F, —CF₃, —CH₂CF₃, —CH₂CCl₃, and the like, and further includes those alkyl groups such as perfluoroalkyl in which all hydrogen atoms are replaced by fluorine atoms.

“Haloalkoxy” refers to substitution of alkoxy groups with 1 to 5 or, in some embodiments, 1 to 3 halo groups, e.g., —OCH₂Cl, —OCH₂F, —OCH₂CH₂Br, —OCH₂CH₂Cl, —OCF₃, and the like.

“Hydroxy” or “hydroxyl” refers to the group —OH.

“Heteroalkyl” means an alkyl radical as defined herein with 1, 2 or 3 substituents independently selected from cyano, —OR^{w}, —NR^{x}R^{y}, —SR^{z}, —S(O)R^{z}, and —S(O)₂R^{z} (where n is 0, 1, or 2), with the understanding that the point of attachment of the heteroalkyl radical is through a carbon atom of the heteroalkyl radical. R^{w} is hydrogen, alkyl, cycloalkyl, cycloalkyl-alkyl, aryl, arylalkyl, alkoxycarbonyl, aryloxycarbonyl, carboxamido, or mono- or di-alkylcarbamoyl. R^{x} is hydrogen, alkyl, cycloalkyl, cycloalkyl-alkyl, aryl or arylalkyl. R^{y} is hydrogen, alkyl, cycloalkyl, cycloalkyl-alkyl, aryl, arylalkyl, alkoxycarbonyl, aryloxycarbonyl, carboxamido, mono- or di-alkylcarbamoyl or alkylsulfonyl. R^{z} is hydrogen, alkyl, cycloalkyl, cycloalkyl-alkyl, aryl, arylalkyl, amino, mono-alkylamino, di-alkylamino, or hydroxyalkyl. Representative examples include, for example, 2-hydroxyethyl, 2,3-dihydroxypropyl, 2-methoxyethyl, benzyloxymethyl, 2-cyanoethyl, and 2-methylsulfonyl-ethyl. For each of the above, R^{w}, R^{x}, R^{y}, and R^{z} can be further substituted by amino, fluorine, alkylamino, di-alkylamino, OH or alkoxy. Additionally, the prefix indicating the number of carbon atoms (e.g., C₁-C₁₀) refers to the total number of carbon atoms in the portion of the heteroalkyl group exclusive of the cyano, —OR^{w}, —NR^{x}R^{y}, —SR^{z}, —S(O)R^{z}, or —S(O)₂R^{z} portions.

“Heteroaryl” refers to an aromatic group of from 1 to 14 carbon atoms and 1 to 6 heteroatoms selected from the group consisting of oxygen, nitrogen, and sulfur and includes a 5- to 18-member ring or ring system that includes a single ring (e.g., imidazolyl) or multiple rings (e.g., benzimidazol-2-yl and benzimidazol-6-yl). For multiple ring systems, including fused, bridged, and spiro ring systems having aromatic and non-aromatic rings, the term “heteroaryl” applies if there is at least one ring heteroatom and the point of attachment is at an atom of an aromatic ring (e.g., 1,2,3,4-tetrahydroquinolin-6-yl and 5,6,7,8-tetrahydroquinolin-3-yl). In one embodiment, the nitrogen and/or the sulfur ring atom(s) of the heteroaryl group are optionally oxidized to provide for the N-oxide (N→O), sulfinyl, or sulfonyl moieties. More specifically the term heteroaryl includes, but is not limited to, pyridyl, furanyl, thienyl, thiazolyl, isothiazolyl, tetrazolyl, triazolyl, imidazolyl, isoxazolyl, pyrrolyl, pyrazolyl, pyridazinyl, pyrimidinyl, benzofuranyl, tetrahydrobenzofuranyl, isobenzofuranyl, benzothiazolyl, benzoisothiazolyl, benzotriazolyl, indolyl, isoindolyl, benzoxazolyl, quinolyl, tetrahydroquinolinyl, isoquinolyl, quinazolinonyl, benzimidazolyl, benzisoxazolyl, or benzothienyl.

“N-linked” refers to nitrogen containing groups in which the point of attachment is to the nitrogen atom of the nitrogen containing group. For example, “N-linked tetrazolyl” is a group in which the point of attachment is to a nitrogen atom of the tetrazolyl group. Similarly, N-linked triazolyl, N-linked imidazolyl, N-linked pyrazolyl and N-linked pyrrolyl are groups in which the point of attachment is to a nitrogen atom of the triazole, imidazole, pyrazole, and pyrrol group, respectively. Similarly, “N-linked imidazolyl” refers to an imidazole in which the point of attachment is to the nitrogen atom.

“Substituted heteroaryl” refers to heteroaryl groups that are substituted with from 1 to 8, or, in some embodiments, 1 to 5, or 1 to 3, or 1 to 2 substituents selected from the group consisting of the substituents defined for substituted aryl.

“Heteroaryloxy” refers to —O-heteroaryl wherein heteroaryl is as defined herein.

“Substituted heteroaryloxy” refers to the group —O-(substituted heteroaryl) wherein heteroaryl is as defined herein.

“Heteroarylthio” refers to the group —S-heteroaryl wherein heteroaryl is as defined herein.

“Substituted heteroarylthio” refers to the group —S-(substituted heteroaryl) wherein heteroaryl is as defined herein.

“Heterocycle” or “heterocyclic” or “heterocyclo” or “heterocycloalkyl” or “heterocyclyl” refers to a saturated or partially saturated cyclic group having from 1 to 14 carbon atoms and from 1 to 6 heteroatoms selected from the group consisting of nitrogen, sulfur, or oxygen and includes single ring and multiple ring systems including fused, bridged, and spiro ring systems. For multiple ring systems having aromatic and/or non-aromatic rings, the term “heterocyclic”, “heterocycle”, “heterocyclo”, “heterocycloalkyl” or “heterocyclyl” applies when there is at least one ring heteroatom and the point of attachment is at an atom of a non-aromatic ring (e.g., 1,2,3,4-tetrahydroquinoline-3-yl, 5,6,7,8-tetrahydroquinoline-6-yl, and decahydroquinolin-6-yl). In one embodiment, the nitrogen and/or sulfur atom(s) of the heterocyclic group are optionally oxidized to provide for the N-oxide, sulfinyl, and sulfonyl moieties. More specifically the heterocyclyl includes, but is not limited to, tetrahydropyranyl, piperidinyl, N-methylpiperidin-3-yl, piperazinyl, N-methylpyrrolidin-3-yl, 3-pyrrolidinyl, 2-pyrrolidon-1-yl, morpholinyl, and pyrrolidinyl. A prefix indicating the number of carbon atoms (e.g., C₃-C₁₀) refers to the total number of carbon atoms in the portion of the heterocyclyl group exclusive of the number of heteroatoms.

“Substituted heterocycle” or “substituted heterocyclic” or “substituted heterocyclo” or “substituted heterocycloalkyl” or “substituted heterocyclyl” refers to heterocyclic groups, as defined herein, that are substituted with from 1 to 5 or, in some embodiments, 1 to 3 of the substituents as defined for substituted cycloalkyl.

“Heterocyclyloxy” refers to the group —O-heterocyclyl wherein heterocyclyl is as defined herein.

“Substituted heterocyclyloxy” refers to the group —O-(substituted heterocyclyl) wherein heterocyclyl is as defined herein.

“Heterocyclylthio” refers to the group —S-heterocycyl wherein heterocyclyl is as defined herein.

“Substituted heterocyclylthio” refers to the group —S-(substituted heterocycyl) wherein heterocyclyl is as defined herein.

Examples of heterocycle and heteroaryl groups include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydroisoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), 1,1-dioxothiomorpholinyl, piperidinyl, pyrrolidine, and tetrahydrofuranyl.

“Nitro” refers to the group —NO₂.

“Oxo” refers to the atom (═O).

“Oxide” refers to products resulting from the oxidation of one or more heteroatoms. Examples include N-oxides, sulfoxides, and sulfones.

“Spirocycloalkyl” refers to a 3- to 10-member cyclic substituent formed by replacement of two hydrogen atoms at a common carbon atom with an alkylene group having 2 to 9 carbon atoms, as exemplified by the following structure wherein the methylene group shown below attached to bonds marked with wavy lines is substituted with a spirocycloalkyl group:

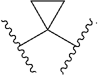

“Sulfonyl” refers to the divalent group —S(O)₂—.

“Substituted sulfonyl” refers to the group —SO₂-alkyl, —SO₂-substituted alkyl, —SO₂-alkenyl, —SO₂-substituted alkenyl, —SO₂-alkynyl, —SO₂-substituted alkynyl, —SO₂-cycloalkyl, —SO₂-substituted cylcoalkyl, —SO₂-aryl, —SO₂-substituted aryl, —SO₂-heteroaryl, —SO₂-substituted heteroaryl, —SO₂-heterocyclic, —SO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein. Substituted sulfonyl includes groups such as methyl-SO₂—, phenyl-SO₂—, and 4-methylphenyl-SO₂—.

“Sulfonyloxy” refers to the group —OSO₂-alkyl, —OSO₂-substituted alkyl, —OSO₂-alkenyl, —OSO₂-substituted alkenyl, —OSO₂-cycloalkyl, —OSO₂-substituted cylcoalkyl, —OSO₂-aryl, —OSO₂-substituted aryl, —OSO₂-heteroaryl, —OSO₂-substituted heteroaryl, —OSO₂-heterocyclic, —OSO₂-substituted heterocyclic, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thioacyl” refers to the groups H—C(S)—, alkyl-C(S)—, substituted alkyl-C(S)—, alkenyl-C(S)—, substituted alkenyl-C(S)—, alkynyl-C(S)—, substituted alkynyl-C(S)—, cycloalkyl-C(S)—, substituted cycloalkyl-C(S)—, aryl-C(S)—, substituted aryl-C(S)—, heteroaryl-C(S)—, substituted heteroaryl-C(S)—, heterocyclic-C(S)—, and substituted heterocyclic-C(S)—, wherein alkyl, substituted alkyl, alkenyl, substituted alkenyl, alkynyl, substituted alkynyl, cycloalkyl, substituted cycloalkyl, aryl, substituted aryl, heteroaryl, substituted heteroaryl, heterocyclic and substituted heterocyclic are as defined herein.

“Thiol” refers to the group —SH.

“Alkylthio” refers to the group —S-alkyl wherein alkyl is as defined herein.

“Substituted alkylthio” refers to the group —S-(substituted alkyl) wherein substituted alkyl is as defined herein.

“Thiocarbonyl” refers to the divalent group —C(S)— which is equivalent to —C(═S)—.

“Thione” refers to the atom (═S).

“Thiocyanate” refers to the group —SCN.

“Compound” and “compounds” as used herein refers to a compound encompassed by the generic formulae disclosed herein, any subgenus of those generic formulae, and any forms of the compounds within the generic and subgeneric formulae, such as an oxide, ester, prodrug, pharmaceutically acceptable salt, or solvate. Unless specified otherwise, the term further includes the racemates, stereoisomers, and tautomers of the compound or compounds.

“Racemates” refers to a mixture of enantiomers.

“Solvate” or “solvates” of a compound refer to those compounds, where compounds are as defined herein, that are bound to a stoichiometric or non-stoichiometric amount of a solvent. Solvates of a compound includes solvates of all forms of the compound such as the oxide, ester, prodrug, or pharmaceutically acceptable salt of the disclosed generic and subgeneric formulae. Preferred solvents are volatile, non-toxic, and/or acceptable for administration to humans.

“Stereoisomer” or “stereoisomers” refer to compounds that differ in the chirality of one or more stereocenters. Stereoisomers include enantiomers and diastereomers. The compounds of this invention may exist in stereoisomeric form if they possess one or more asymmetric centers or a double bond with asymmetric substitution and, therefore, can be produced as individual stereoisomers or as mixtures. Unless otherwise indicated, the description is intended to include individual stereoisomers as well as mixtures. The methods for the determination of stereochemistry and the separation of stereoisomers are well-known in the art (see discussion in Chapter 4 of Advanced Organic Chemistry, 4th ed., J. March, John Wiley and Sons, New York, 1992).

“Tautomer” refers to alternate forms of a compound that differ in the position of a proton, such as enol-keto and imine-enamine tautomers, or the tautomeric forms of heteroaryl groups containing a ring atom attached to both a ring —NH— moiety and a ring ═N— moiety such as pyrazoles, imidazoles, benzimidazoles, triazoles, and tetrazoles.

“Prodrug” refers to any derivative of a compound of the embodiments that is capable of directly or indirectly providing a compound of the embodiments or an active metabolite or residue thereof when administered to a patient. Prodrugs of a compound of the present invention are prepared by modifying functional groups present in the compound in such a way that the modifications may be cleaved in vivo to release the parent compound, or an active metabolite. For example, prodrugs include compounds wherein a hydroxy, amino, or sulfhydryl group in a compound I is bonded to any group that may be cleaved in vivo to regenerate the free hydroxyl, amino, or sulfhydryl group, respectively. Particularly favored derivatives and prodrugs are those that increase the bioavailability of the compounds of the embodiments when such compounds are administered to a patient (e.g., by allowing an orally administered compound to be more readily absorbed into the blood) or which enhance delivery of the parent compound to a biological compartment (e.g., the brain or lymphatic system) relative to the parent species. Prodrugs include ester, amide, and carbamate (e.g., N,N-dimethylaminocarbonyl) forms of hydroxy functional groups of compounds of the invention. Examples of ester prodrugs include formate, acetate, propionate, butyrate, acrylate, and ethylsuccinate derivatives. An general overview of prodrugs is provided in T Higuchi and V Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Pharmaceutically acceptable salt” refers to pharmaceutically acceptable salts derived from a variety of organic and inorganic counter ions well known in the art and includes, by way of example only, sodium, potassium, calcium, magnesium, ammonium, and tetraalkylammonium. When the molecule contains a basic functionality, acid addition salts of organic or inorganic acids, such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, oxalic acid, 4-toluenesulfonic acid, camphorsulfonic acid, methanesulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Salts can also be formed when an acidic proton present in the parent compound is either replaced by a metal ion, e.g., an alkali metal ion, an alkaline earth ion, or an aluminum ion; or coordinates with an organic base such as ethanolamine, diethanolamine, triethanolamine, trimethylamine, N-methylglucamine, and the like. Pharmaceutically acceptable salts are suitable for administration in a patient and possess desirable pharmacological properties. Suitable salts further include those described in P. Heinrich Stahl, Camille G. Wermuth (Eds.), Handbook of Pharmaceutical Salts Properties, Selection, and Use; 2002.

Unless indicated otherwise, the nomenclature of substituents that are not explicitly defined herein are arrived at by naming the terminal portion of the functionality followed by the adjacent functionality toward the point of attachment. For example, the substituent “arylalkyloxycabonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups with two other substituted aryl groups are limited to -substituted aryl-(substituted aryl)-substituted aryl.

Similarly, it is understood that the above definitions are not intended to include impermissible substitution patterns (e.g., methyl substituted with 5 fluoro groups). Such impermissible substitution patterns are well known to the skilled artisan.

The terms “optional” or “optionally” as used throughout the specification means that the subsequently described event or circumstance may but need not occur, and that the description includes instances where the event or circumstance occurs and instances in which it does not. For example, “heterocyclo group optionally mono- or di-substituted with an alkyl group” means that the alkyl may but need not be present, and the description includes situations where the heterocyclo group is mono- or disubstituted with an alkyl group and situations where the heterocyclo group is not substituted with the alkyl group.

Turning next to the compositions of the invention, the term “pharmaceutically acceptable carrier or excipient” means a carrier or excipient that is useful in preparing a pharmaceutical composition that is generally safe, and possesses acceptable toxicities. Acceptable carriers or excipients include those that are acceptable for veterinary use as well as human pharmaceutical use. A “pharmaceutically acceptable carrier or excipient” as used in the specification and claims includes both one and more than one such carrier or excipient.

With reference to the methods of the present invention, the following terms are used with the noted meanings:

The terms “treating” or “treatment” of a disease includes:

(1) preventing or reducing the risk of developing the disease, i.e., causing the clinical symptoms of the disease not to develop in a mammal that may be exposed to or predisposed to the disease but does not yet experience or display symptoms of the disease,

(2) inhibiting the disease, i.e., arresting or reducing the development of the disease or its clinical symptoms, or

(3) relieving the disease, i.e., causing regression of the disease or its clinical symptoms.

A preferred embodiment of the invention is treatment of a disease that consists of relieving the disease.

The term “diagnosing” refers to determining the presence or absence of a particular disease or condition. Additionally, the term refers to determining the level or severity of a particular disease or condition, as well as monitoring of the disease or condition to determine its response to a particular therapeutic regimen.

The term “therapeutically effective amount” means the amount of the subject compound that will elicit the biological or medical response of a tissue, system, animal or human that is being sought by the researcher, veterinarian, medical doctor or other clinician. “A therapeutically effective amount” includes the amount of a compound that, when administered to a mammal for treating a disease, is sufficient to effect such treatment for the disease. The “therapeutically effective amount” will vary depending on the compound, the disease and its severity and the age, weight, etc., of the mammal to be treated.

“Patient” refers to mammals and includes humans and non-human mammals. Examples of patients include, but are not limited to mice, rats, hamsters, guinea pigs, pigs, rabbits, cats, dogs, goats, sheep, cows, and humans.

The term “mammal” includes, without limitation, humans, domestic animals (e.g., dogs or cats), farm animals (cows, horses, or pigs), and laboratory animals (mice, rats, hamsters, guinea pigs, pigs, rabbits, dogs, or monkeys).

The term “insulin resistance” can be defined generally as a disorder of glucose metabolism. More specifically, insulin resistance can be defined as the diminished ability of insulin to exert its biological action across a broad range of concentrations producing less than the expected biologic effect (see, e.g., Reaven G M, J. Basic & Clin. Phys. & Pharm. (1998) 9:387-406 and Flie J, Ann. Rev. Med. (1983) 34:145-60). Insulin resistant persons have a diminished ability to properly metabolize glucose and respond poorly, if at all, to insulin therapy. Manifestations of insulin resistance include insufficient insulin activation of glucose uptake, oxidation and storage in muscle and inadequate insulin repression of lipolysis in adipose tissue and of glucose production and secretion in liver. Insulin resistance can cause or contribute to polycystic ovarian syndrome, impaired glucose tolerance, gestational diabetes, metabolic syndrome, hypertension, obesity, atherosclerosis and a variety of other disorders. Eventually, the insulin resistant individuals can progress to a point where a diabetic state is reached.

The term “diabetes mellitus” or “diabetes” means a disease or condition that is generally characterized by metabolic defects in production and utilization of glucose that result in the failure to maintain appropriate blood sugar levels in the body. The result of these defects is elevated blood glucose, referred to as “hyperglycemia.” Two major forms of diabetes are Type I diabetes and Type II diabetes. As described above, Type I diabetes is generally the result of an absolute deficiency of insulin, the hormone that regulates glucose utilization. Type II diabetes often occurs in the face of normal, or even elevated levels of insulin and can result from the inability of tissues to respond appropriately to insulin. Most Type II diabetic patients are insulin resistant and have a relative deficiency of insulin, in that insulin secretion can not compensate for the resistance of peripheral tissues to respond to insulin. In addition, many Type II diabetics are obese. Other types of disorders of glucose homeostasis include impaired glucose tolerance, which is a metabolic stage intermediate between normal glucose homeostasis and diabetes, and gestational diabetes mellitus, which is glucose intolerance in pregnancy in women with no previous history of Type I or Type II diabetes.

The term “metabolic syndrome” refers to a cluster of metabolic abnormalities including abdominal obesity, insulin resistance, glucose intolerance, diabetes, hypertension and dyslipidemia. These abnormalities are known to be associated with an increased risk of vascular events.

The term “abdominal obesity” is defined by a cutoff point of waist circumference ≧102 cm in men and ≧80 cm in women, as recommended by the third report of the national cholesterol education program expert panel on detection, evaluation, and treatment of high blood cholesterol in adults (NCEP/ATP Panel III).

The guidelines for diagnosis of Type II diabetes, impaired glucose tolerance, and gestational diabetes have been outlined by the American Diabetes Association (see, e.g., The Expert Committee on the Diagnosis and Classification of Diabetes Mellitus, Diabetes Care, (1999) Vol. 2 (Suppl 1):S5-19).

The term “secretagogue” means a substance or compound that stimulates secretion. For example, an insulin secretagogue is a substance or compound that stimulates secretion of insulin.

The term “symptom” of diabetes, includes, but is not limited to, polyuria, polydipsia, and polyphagia, as used herein, incorporating their common usage. For example, “polyuria” means the passage of a large volume of urine during a given period; “polydipsia” means chronic, excessive thirst; and “polyphagia” means excessive eating. Other symptoms of diabetes include, e.g., increased susceptibility to certain infections (especially fungal and staphylococcal infections), nausea, and ketoacidosis (enhanced production of ketone bodies in the blood).

The term “complication” of diabetes includes, but is not limited to, microvascular complications and macrovascular complications. Microvascular complications are those complications that generally result in small blood vessel damage. These complications include, e.g., retinopathy (the impairment or loss of vision due to blood vessel damage in the eyes); neuropathy (nerve damage and foot problems due to blood vessel damage to the nervous system); and nephropathy (kidney disease due to blood vessel damage in the kidneys). Macrovascular complications are those complications that generally result from large blood vessel damage. These complications include, e.g., cardiovascular disease and peripheral vascular disease. Cardiovascular disease refers to diseases of blood vessels of the heart. See, e.g., Kaplan R M, et al., “Cardiovascular diseases” in Health and Human Behavior, pp. 206-242 (McGraw-Hill, New York 1993). Cardiovascular disease is generally one of several forms, including, e.g., hypertension (also referred to as high blood pressure), coronary heart disease, stroke, and rheumatic heart disease. Peripheral vascular disease refers to diseases of any of the blood vessels outside of the heart. It is often a narrowing of the blood vessels that carry blood to leg and arm muscles.

The term “atherosclerosis” encompasses vascular diseases and conditions that are recognized and understood by physicians practicing in the relevant fields of medicine. Atherosclerotic cardiovascular disease, coronary heart disease (also known as coronary artery disease or ischemic heart disease), cerebrovascular disease and peripheral vessel disease are all clinical manifestations of atherosclerosis and are therefore encompassed by the terms “atherosclerosis” and “atherosclerotic disease”.

The term “antihyperlipidemic” refers to the lowering of excessive lipid concentrations in blood to desired levels.

The term “modulate” refers to the treating, prevention, suppression, enhancement or induction of a function or condition. For example, compounds can modulate Type II diabetes by increasing insulin in a human, thereby suppressing hyperglycemia.

The term “triglyceride(s)” (“TGs”), as used herein, incorporates its common usage. TGs consist of three fatty acid molecules esterified to a glycerol molecule. TGs serve to store fatty acids that are used by muscle cells for energy production or are taken up and stored in adipose tissue.

Because cholesterol and TGs are water insoluble, they must be packaged in special molecular complexes known as “lipoproteins” in order to be transported in the plasma. Lipoproteins can accumulate in the plasma due to overproduction and/or deficient removal. There are at least five distinct lipoproteins differing in size, composition, density, and function. In the cells of the small intestine, dietary lipids are packaged into large lipoprotein complexes called “chylomicrons”, which have a high TG and low-cholesterol content. In the liver, TG and cholesterol esters are packaged and released into plasma as TG-rich lipoprotein called very low density lipoprotein (“VLDL”), whose primary function is the endogenous transport of TGs made in the liver or released by adipose tissue. Through enzymatic action, VLDL can be either reduced and taken up by the liver, or transformed into intermediate density lipoprotein (“IDL”). IDL, is in turn, either taken up by the liver, or is further modified to form low density lipoprotein (“LDL”). LDL is either taken up and broken down by the liver, or is taken up by extrahepatic tissue. High density lipoprotein (“HDL”) helps remove cholesterol from peripheral tissues in a process called reverse cholesterol transport.

The term “dyslipidemia” refers to abnormal levels of lipoproteins in blood plasma including both depressed and/or elevated levels of lipoproteins (e.g., elevated levels of LDL and/or VLDL, and depressed levels of HDL).

The term “hyperlipidemia” includes, but is not limited to, the following:

(1) Familial Hyperchylomicronemia, a rare genetic disorder that causes a deficiency in an enzyme, LP lipase, that breaks down fat molecules. The LP lipase deficiency can cause the accumulation of large quantities of fat or lipoproteins in the blood;

(2) Familial Hypercholesterolemia, a relatively common genetic disorder caused where the underlying defect is a series of mutations in the LDL receptor gene that result in malfunctioning LDL receptors and/or absence of the LDL receptors. This brings about ineffective clearance of LDL by the LDL receptors resulting in elevated LDL and total cholesterol levels in the plasma;

(3) Familial Combined Hyperlipidemia, also known as multiple lipoprotein-type hyperlipidemia is an inherited disorder where patients and their affected first-degree relatives can at various times manifest high cholesterol and high triglycerides. Levels of HDL cholesterol are often moderately decreased;

(4) Familial Defective Apolipoprotein B-100 is a relatively common autosomal dominant genetic abnormality. The defect is caused by a single nucleotide mutation that produces a substitution of glutamine for arginine, which can cause reduced affinity of LDL particles for the LDL receptor. Consequently, this can cause high plasma LDL and total cholesterol levels;

(5) Familial Dysbetaliproteinemia, also referred to as Type III Hyperlipoproteinemia, is an uncommon inherited disorder resulting in moderate to severe elevations of serum TG and cholesterol levels with abnormal apolipoprotein E function. HDL levels are usually normal; and

(6) Familial Hypertriglyceridemia, is a common inherited disorder in which the concentration of plasma VLDL is elevated. This can cause mild to moderately elevated TG levels (and usually not cholesterol levels) and can often be associated with low plasma HDL levels.

Risk factors for hyperlipidemia include, but are not limited to, the following: (1) disease risk factors, such as a history of Type I diabetes, Type II diabetes, Cushing's syndrome, hypothyroidism and certain types of renal failure; (2) drug risk factors, which include, birth control pills; hormones, such as estrogen, and corticosteroids; certain diuretics; and various β-blockers; (3) dietary risk factors include dietary fat intake per total calories greater than 40%; saturated fat intake per total calories greater than 10%; cholesterol intake greater than 300 mg per day; habitual and excessive alcohol use; and obesity.

The terms “obese” and “obesity” refers to, according to the World Health Organization, a Body Mass Index (“BMI”) greater than 27.8 kg/m² for men and 27.3 kg/m² for women (BMI equals weight (kg)/height (m²)). Obesity is linked to a variety of medical conditions including diabetes and hyperlipidemia. Obesity is also a known risk factor for the development of Type II diabetes (see, e.g., Barrett-Conner E, Epidemol. Rev. (1989) 11:172-181; and Knowler, et al., Am. J. Clin. Nutr. (1991) 53:1543-1551).

The term “pancreas” refers to a gland organ in the digestive and endocrine system of vertebrates, including mammals. The pancreas secretes both digestive enzymes and hormones such as insulin, GLP-1 and GIP as well as other hormones.

The term “islet” or “islet of Langerhans” refers to endocrine cells of the pancreas that are grouped together in islets and secrete insulin and other hormones.

The term “beta cell” refers to cells found in the islet of Langerhans that secrete insulin, amylin, and other hormones.

The term “endocrine cell” refers to cells that secrete hormones into the blood stream. Endocrine cells are found various glands and organ systems of the body including the pancreas, intestines, and other organs.

The term “L cell” refers to gut endocrine cells that produce GLP-1.

The term “K cell” refers to gut endocrine cells that produce GIP.

The term “incretin” refers to a group of hormones that increases insulin secretion in response to food intake. Incretins include GLP-1 and GIP.

The term “insulin” refers to a polypeptide hormone that regulates glucose metabolism. Insulin binds to insulin receptors in insulin sensitive cells and mediates glucose uptake. Insulin is used to treat Type I diabetes and may be used to treat Type II diabetes.

The term “GLP-1” or “glucagon-like peptide” is a peptide hormone primarily produced by L cells. GLP-1 increases insulin secretion, decreases glucagon secretion, increases beta cell mass and insulin gene expression, inhibits acid secretion and gastric emptying in the stomach, and decreases food intake by increasing satiety.

The term “GIP” or “gastric inhibitory peptide” or “glucose dependent insulinotropic polypeptide” refers to a peptide hormone produced primarily by K cells. GIP stimulates insulin secretion. GIP also has significant effects on lipid metabolism.

The term “cAMP” or “cyclic AMP” or “cyclic adenosine monophosphate” refers to an intracellular signaling molecule involved in many biological processes, including glucose and lipid metabolism.

The term “agonist” refers to a compound that binds to a receptor and triggers a response in a cell. An agonist mimics the effect of an endogenous ligand, a hormone for example, and produces a physiological response similar to that produced by the endogenous ligand.

The term “partial agonist” refers to a compound that binds to a receptor and triggers a partial response in a cell. A partial agonist produces only a partial physiological response of the endogenous ligand.

The present invention derives from the discovery of compounds that act as agonists of GPR119 using a cell-based screen. A stable CHO cell line expressing GPR119 under the control of the CMV promoter was used and cAMP levels were measured in the cells using a homogeneous time resolved fluorescence assay. With a parental CHO cell line as a control, increased cAMP levels could be measured and compounds identified that, like exenatide, raise cAMP in cells. Since elevated intracellular cAMP levels in the beta cell increase insulin secretion in a glucose dependant manner (see Biological Example 2), the present invention is useful for the treatment of, inter alia, Type II diabetes and other diseases associated with poor glycemic control. Additionally, the islet specific expression of the receptor for the novel agonists of the present invention also make the present invention useful for the diagnosis of, inter alia, diabetes and other diseases associated with beta cell health.

The compounds of the present invention are represented by by Formula (I), (II) or (III) as shown below.

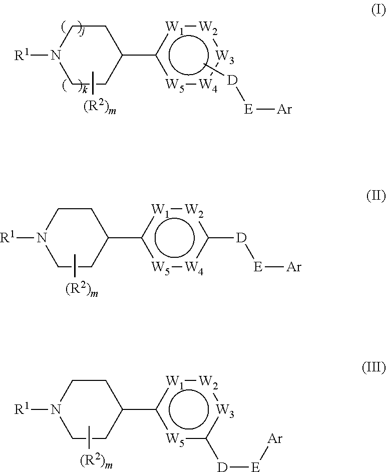

In Formulas (I), (II) and (III), W₁, W₂, W₃, W₄ and W₅ are independently selected from the group consisting of CR³, and N, provided that only zero, one, two, or three of W₁, W₂, W₃, W₄ and W₅ is N. D, and E are independently selected from the group consisting of a bond, —(CHR⁴)ₚ—, —C(O)—, —O—, —S—, —S(O)—, —S(O)₂—, and —NR⁵—, provided that one of D or E is —(CHR⁴)ₚ— or —C(O)—. The subscript p is 0, 1, or 2. The subscript j is 0, 1, or 2. The subscript k is 0, 1, or 2. The subscript m is 0, 1, 2, 3, or 4. Ar is a 5- to 10-membered aryl or heteroaryl group, optionally substituted with from 1 to 5 R⁶ groups.

Turning next to R¹, R¹ is selected from the group consisting of H, C₁₋₁₀alkyl, C₁₋₁₀substituted alkyl, C₃₋₇cycloalkyl, C₂₋₁₀alkenyl, C₂₋₁₀alkynyl, —X¹—COR^{a}, —X¹—CO₂R^{a}, —X¹—CONR^{a}R^{b}, —SO₂R^{a}, a 4- to 7-membered heterocyclyl group, aryl and a 5- to 10-membered heteroaryl group, wherein each of said cycloalkyl group, heterocyclyl group, aryl group and heteroaryl group is optionally substituted with from 1 to 4 substituents independently selected from the group consisting of halo, C₁₋₁₀alkyl, C₁₋₁₀substituted alkyl, C₃₋₇cycloalkyl, C₂₋₁₀alkenyl, C₂₋₁₀alkynyl, aryl, heteroaryl, —CN, —NR^{a}COR^{b}, —NR^{a}CONR^{a}R^{b}, —NO₂, —OR^{a}, —NR^{a}R^{b}, —COR^{a}, —CO₂R^{a}, —CONR^{a}R^{b}, —SR^{a}, —S(O)R^{a}, —S(O)₂R^{a}, —NR^{a}S(O)₂R^{b}, and —SO₂NR^{a}R^{b}, or optionally R^{a} and R^{b} are combined to form a 4-, 5- or 6-membered ring, and X¹ is selected from the group consisting of a bond, C₁₋₄alkylene, C₂₋₆alkenylene, C₂₋₆alkynylene, —C(O)—, and —C(O)—(CH₂)₁₋₄—, wherein the aliphatic portions of X¹ are optionally substituted with 1 to 3 members selected from the group consisting of halo, C₁₋₄alkyl, C₁₋₄substituted alkyl and C₁₋₄haloalkyl.

Each R² is independently selected from the group consisting of H, halo, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b}, and wherein when the subscript m is 2 and R² is alkyl or substituted alkyl, the two R² members can optionally cyclize to form a ring.

Next, R³ is selected from the group consisting of H, halo, cyano, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b}.

Next, each R⁴ is independently selected from the group consisting of H, halo, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b}.

R⁵ is selected from the group consisting of H, C₁₋₅alkyl, and C₁₋₅substituted alkyl;

Turning next to R⁶, each R⁶ is independently selected from the group consisting of H, halo, C₁₋₁₀alkyl, C₁₋₁₀substituted alkyl, C₃₋₇cycloalkyl, C₂₋₁₀alkenyl, C₂₋₁₀alkynyl, CN, NO₂, —OR^{a}, —NR^{a}R^{b}, —COR^{a}, —CO₂R^{a}, —CONR^{a}R^{b}, —NR^{a}COR^{b}, —NR^{a}CO₂R^{b}, —NR^{a}CONR^{a}R^{b}, —SR^{a}, —S(O)R^{a}, —S(O)₂R^{a}, —NR^{a}S(O)R^{b}, —NR^{a}S(O)₂R^{b}, —SO₂NR^{a}R^{b}, a 4- to 7-membered heterocyclyl group, aryl and a 5- to 10-membered heteroaryl group, wherein each of said heterocyclyl groups, said aryl and heteroaryl groups are optionally substituted with from 1 to 4 substituents independently selected from the group consisting of halo, oxo, C₁₋₄alkyl, C₁₋₄haloalkyl, C₃₋₇cycloalkyl, CN, NO₂, —OR^{a}, —NR^{a}R^{b}, —COR^{a}, —CO₂R^{a}, —CONR^{a}R^{b}, —NR^{a}COR^{b}, —NR^{a}CO₂R^{b}, —NR^{a}CONR^{a}R^{b}, —SR^{a}, —S(O)R^{a}, —S(O)₂R^{a}, —NR^{a}SO₂R^{b}, and —SO₂NR^{a}R^{b}, and optionally R^{a} and R^{b} are combined to form a 4-, 5- or 6-membered ring.

In the compounds of Formula (I), (II) or (III), each R^{a} and R^{b} is independently selected from the group consisting of hydrogen, C₁₋₁₀alkyl, C₁₋₁₀haloalkyl, C₃₋₁₀cycloalkyl, heterocyclyl, C₂₋₁₀alkenyl, C₂₋₁₀alkynyl, aryl, substituted aryl, 5- to 6-membered heteroaryl, 5- to 6-membered substituted heteroaryl, and arylC₁₋₄alkyl; and wherein the aliphatic portions of each of said R^{a} and R^{b} is optionally substituted with from 1 to 3 members selected from the group consisting of halo, —ORⁿ, —OCORⁿ, —OC(O)N(Rⁿ)₂, —SRⁿ, —S(O)Rⁿ, —S(O)₂Rⁿ, —S(O)₂N(Rⁿ)₂, —NR²S(O)₂Rⁿ, —C(O)N(Rⁿ)₂, —C(O)Rⁿ, —NRⁿC(O)Rⁿ, —NRⁿC(O)N(Rⁿ)₂, —CO₂Rⁿ, —NRⁿCO₂Rⁿ, —CN, —NO₂, —N(Rⁿ)₂ and —NRⁿS(O)₂N(Rⁿ)₂, wherein each Rⁿ is independently hydrogen or an unsubstituted C₁₋₆alkyl.

The compounds of Formula (I), (II) or (III) provided herein also include any pharmaceutically acceptable salts, solvates, stereoisomers and esters of the compounds as well as any isotopically labeled isomers thereof. In general, the compounds useful in the methods described herein are those compound of the formula above, wherein the molecular weight of the compound is less than 1200, more preferably less than about 1000, still more preferably less than about 800 and still more preferably from about 200 to about 600.

In one embodiment, two of W₁, W₂, W₃, W₄, and W₅ is N. In another embodiment, one of W₁, W₂, W₃, W₄, and W₅ is N.

In another embodiment, R¹ is selected from the group consisting of —X¹—COR^{a}, —X¹—CO₂R^{a}, —X¹—CONR^{a}R^{b}, SO₂R^{a}, aryl, heteroaryl, substituted aryl and substituted heteroaryl.

Turning next to D and E, preferred embodiments are compounds wherein D is —CH₂— or —O—. Alternatively, E is —CH₂— or —O—. Compounds in which D is —CH₂— and E is —O— are also preferred embodiments. Additionally, compounds in which E is —CH₂— and D is —O— are also preferred embodiments.

For the compounds of Formula (I), (II) or (III), in one group of embodiments, Ar is selected from the group consisting of phenyl, pyridyl, pyrimidinyl, pyrazinyl, pyridazinyl, triazinyl, substituted phenyl, substituted pyridyl, substituted pyrimidinyl, substituted pyrazinyl, substituted pyridazinyl, and substituted triazinyl. When Ar is substituted aryl, the aryl is independently substituted with one or two R⁶ groups.

Preferred R⁶ groups of Formula (I), (II) or (III) are independently selected from the group consisting of halo, C₁₋₅alkyl, C₁₋₅haloalkyl, —SOR^{a}, —SO₂R^{a}, and 5-membered heteroaryl group. Even more preferred, the R⁶ group is independently selected from the group consisting of fluoro, —CH₃, —S(O)₂CH₃, N-linked tetrazolyl, N-linked triazolyl, N-linked imidazolyl, N-linked pyrazolyl and N-linked pyrrolyl.

In another embodiment, provided is a compound wherein zero, one or two of W₁, W₂, W₃, W₄, and W₅ is N; D and E are independently —CH₂— or —O—; Ar is selected from the group consisting of phenyl, pyridyl, pyrimidinyl, pyrazinyl, pyridazinyl, and triazinyl; R₁ is selected from the group consisting of phenyl, pyridyl, pyrimidinyl, pyrazinyl, pyridazinyl, triazinyl, substituted phenyl, substituted pyridyl, substituted pyrimidinyl, substituted pyrazinyl, substituted pyridazinyl, and substituted triazinyl, and wherein when Ar is substituted, Ar is independently substituted with one or two R⁶ groups. In some aspects, R⁶ is selected from the group consisting of fluoro, —CH₃, —S(O)₂CH₃, N-linked tetrazolyl, N-linked triazolyl, and N-linked imidazolyl, N-linked pyrazolyl and N-linked pyrrolyl.

Preparation of Compounds of the Invention

The compounds of the present invention can be prepared in a number of ways familiar to one skilled in the art of organic chemistry synthesis. The synthetic route of compounds in the present invention is not limited to the methods outlined herein or as provided in the Examples. Individual compounds may require manipulation of the conditions in order to accommodate various functional groups and may require appropriate use of protecting groups. Purification, if necessary, can be accomplished on a silica gel column eluted with the appropriate organic solvent system. Also, reverse phase HPLC or recrystallization may be employed.

Compositions and Methods of Treatment

In accordance with the present invention methods of treating a disease or condition selected from the group consisting of Type I diabetes, Type II diabetes and metabolic syndrome are provided. The method comprises administering to a subject in need of such treatment an effective amount of a compound of the present invention.

In another aspect, methods of raising intracellular levels of cyclic AMP (cAMP) in a cell expressing GPR119 are provided. The method comprises exposing a cell that expresses GPR119 to a compound of the invention. Cyclic AMP levels are determined by the methods disclosed in the Example sections herein.

In one embodiment, the cell that expresses GPR119 is a pancreatic cell, an islet cell, or a beta cell, an intestinal endocrine cell, an L cell or a K cell.

Another aspect of the invention provides a method of stimulating insulin production in a mammal, in particular a human. The method comprises administering an effective amount of a compound of the invention to the mammal. In response to administration of a compound to the subject, insulin is produced by the beta cells. Biological Example 2 provides detailed methods by which a skilled artisan can measure insulin secretion in laboratory animals in response to administration of a compound of the invention.

In another aspect, the invention provides a method of stimulating insulin secretion in a mammal, in particular a human. The method comprises administering an effective amount of a compound of the invention to the mammal. In response to administration of a compound to the subject, insulin is secreted into the blood stream by the beta cells. Biological Example 2 provides methods of determining insulin secretion in rats.

A further aspect of the invention provides a method of stimulating glucose-dependent insulin secretion in a mammal, in particular a human. The method comprises administering an effective amount of a compound of the invention to the mammal. After administration to the subject, insulin is secreted into the blood stream by the beta cells in a glucose-dependent manner. Biological Example 3 provides methods that show the blood glucose lowering effects of the compounds of the invention.

In another embodiment, the invention provides methods of lowering blood glucose in a mammal, preferably a human. The method comprises administering an effective amount of a compound of the invention to the mammal. In response to administration of a compound to the subject, blood glucose levels are lowered. The method further comprises steps to measure blood glucose levels before and after administration of a compound of the invention. Blood glucose levels are easily measured by numerous commercially available glucose monitoring devices that measure blood glucose from samples of blood or urine. Blood glucose can also be measured by commercially available glucometers that do not require blood or urine samples. Biological Examples 2 and 5 provide methods that teach how to measure improvements in diabetes paramaters, including blood glucose monitoring.

Another aspect of the invention provides a method of stimulating incretin production in a mammal, in particular a human. The method comprises administering an effective amount of a compound of the invention to the mammal. In response to administration of a compound to the subject, glucagon-like peptide 1 and glucose-dependent insulinotropic polypeptide is produced by the intestinal endocrine cells. Biological Example 4 provides detailed methods by which a skilled artisan can measure incretin production in laboratory animals in response to administration of a compound of the invention.

Combination Therapy

As noted above, the compounds of the present invention will, in some instances, be used in combination with other therapeutic agents to bring about a desired effect. Selection of additional agents will, in large part, depend on the desired target therapy (see, e.g., Turner N, et al., Prog. Drug Res. (1998) 51:33-94; Haffner S, Diabetes Care (1998) 21:160-178; and DeFronzo R, et al. (eds.), Diabetes Reviews (1997) Vol. 5 No. 4). A number of studies have investigated the benefits of combination therapies with oral agents (see, e.g., Mahler R, J. Clin. Endocrinol. Metab. (1999) 84:1165-71; United Kingdom Prospective Diabetes Study Group: UKPDS 28, Diabetes Care (1998) 21:87-92; Bardin C W (ed.), Current Therapy in Endocrinology and Metabolism, 6th Ed. (Mosby—Year Book, Inc., St. Louis, Mo. 1997); Chiasson J, et al., Ann. Intern. Med. (1994) 121:928-935; Coniff R, et al., Clin. Ther. (1997) 19:16-26; Coniff R, et al., Am. J. Med. (1995) 98:443-451; and Iwamoto Y, et al., Diabet. Med. (1996) 13:365-370; Kwiterovich P, Am. J. Cardiol (1998) 82(12A):3U-17U). These studies indicate that diabetes modulation can be further improved by the addition of a second agent to the therapeutic regimen. Combination therapy includes administration of a single pharmaceutical dosage formulation that contains a compound having the general structure of Formula (I), (II) or (III) and one or more additional active agents, as well as administration of a compound of Formula (I), (II) or (III) and each active agent in its own separate pharmaceutical dosage formulation. For example, a compound of Formula (I), (II) or (III) and a DPP4 inhibitor can be administered to the human subject together in a single oral dosage composition, such as a tablet or capsule, or each agent can be administered in separate oral dosage formulations. Where separate dosage formulations are used, a compound of Formula (I), (II) or (III) and one or more additional active agents can be administered at essentially the same time (i.e., concurrently), or at separately staggered times (i.e., sequentially). Combination therapy is understood to include all these regimens.

An example of combination therapy can be seen in modulating (preventing the onset of the symptoms or complications associated with diabetes or treating, preventing or reducing or the risk of developing diabetes and its related symptoms, complications, and disorders), wherein the compounds of Formula (I), (II) or (III) can be effectively used in combination with, for example, biguanides (such as metformin); thiazolidinediones (such as ciglitazone, pioglitazone, troglitazone, and rosiglitazone); dipeptidyl-peptidase-4 (“DPP4”) inhibitors (such as vildagliptin and sitagliptin); glucagonlike peptide-1 (“GLP-1”) receptor agonists (such as exanatide) (or GLP-1 mimetics); PPAR gamma agonists or partial agonists; dual PPAR alpha, PPAR gamma agonists or partial agonists; dual PPAR delta, PPAR gamma agonists or partial agonists; pan PPAR agonists or partial agonists; dehydroepiandrosterone (also referred to as DHEA or its conjugated sulphate ester, DHEA-SO₄); antiglucocorticoids; TNFα inhibitors; α-glucosidase inhibitors (such as acarbose, miglitol, and voglibose); sulfonylureas (such as chlorpropamide, tolbutamide, acetohexamide, tolazamide, glyburide, gliclazide, glynase, glimepiride, and glipizide); pramlintide (a synthetic analog of the human hormone amylin); other insulin secretogogues (such as repaglinide, gliquidone, and nateglinide); insulin (or insulin mimetics); glucagon receptor antagonists; gastric inhibitory peptide (“GIP”); or GIP mimetics; as well as the active agents discussed below for treating obesity, hyperlipidemia, atherosclerosis and/or metabolic syndrome.

Another example of combination therapy can be seen in treating obesity or obesity-related disorders, wherein the compounds of Formula (I), (II) or (III) can be effectively used in combination with, for example, phenylpropanolamine, phenteramine; diethylpropion; mazindol; fenfluramine; dexfenfluramine; phentiramine, β-3 adrenoceptor agonist agents; sibutramine; gastrointestinal lipase inhibitors (such as orlistat); and leptins. Other agents used in treating obesity or obesity-related disorders wherein the compounds of Formula (I), (II) or (III) can be effectively used in combination with, for example, cannabinoid-1 (“CB-1”) receptor antagonists (such as rimonabant); PPAR delta agonists or partial agonists; dual PPAR alpha, PPAR delta agonists or partial agonists; dual PPAR delta, PPAR gamma agonists or partial agonists; pan PPAR agonists or partial agonists; neuropeptide Y; enterostatin; cholecytokinin; bombesin; amylin; histamine H₃ receptors; dopamine D₂ receptors; melanocyte stimulating hormone; corticotrophin releasing factor; galanin; and gamma amino butyric acid (GABA).

Still another example of combination therapy can be seen in modulating hyperlipidemia (e.g. treating hyperlipidemia and its related complications), wherein the compounds of Formula (I), (II) or (III) can be effectively used in combination with, for example, statins (such as atorvastatin, fluvastatin, lovastatin, pravastatin, and simvastatin), CETP inhibitors (such as torcetrapib); a cholesterol absorption inhibitor (such as ezetimibe); PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; fenofibric acid derivatives (such as gemfibrozil, clofibrate, fenofibrate, and bezafibrate); bile acid-binding resins (such as colestipol or cholestyramine); nicotinic acid; probucol; betacarotene; vitamin E; or vitamin C.

A further example of combination therapy can be seen in modulating atherosclerosis, wherein a compound of Formula (I), (II) or (III) is administered in combination with one or more of the following active agents: an antihyperlipidemic agent; a plasma HDL-raising agent; an antihypercholesterolemic agent, such as a cholesterol biosynthesis inhibitor, e.g., an hydroxymethylglutaryl (HMG) CoA reductase inhibitor (also referred to as statins, such as lovastatin, simvastatin, pravastatin, fluvastatin, and atorvastatin); an HMG-CoA synthase inhibitor; a squalene epoxidase inhibitor; or a squalene synthetase inhibitor (also known as squalene synthase inhibitor); an acyl-coenzyme A cholesterol acyltransferase (ACAT) inhibitor, such as melinamide; probucol; nicotinic acid and the salts thereof and niacinamide; a cholesterol absorption inhibitor, such as β-sitosterol; a bile acid sequestrant anion exchange resin, such as cholestyramine, colestipol or dialkylaminoalkyl derivatives of a cross-linked dextran; an LDL receptor inducer; fibrates, such as clofibrate, bezafibrate, fenofibrate, and gemfibrizol; vitamin B₆ (also known as pyridoxine) and the pharmaceutically acceptable salts thereof, such as the HCl salt; vitamin B₁₂ (also known as cyanocobalamin); vitamin B₃ (also known as nicotinic acid and niacinamide); anti-oxidant vitamins, such as vitamin C and E and beta carotene; a β-blocker; an angiotensin II antagonist; an angiotensin converting enzyme inhibitor; PPAR alpha agonists or partial agonists; PPAR delta agonists or partial agonists; PPAR gamma agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; and a platelet aggregation inhibitor, such as fibrinogen receptor antagonists (i.e., glycoprotein IIb/IIIa fibrinogen receptor antagonists) and aspirin. As noted above, the compounds of Formula (I), (II) or (III) can be administered in combination with more than one additional active agent, for example, a combination of a compound of Formula (I), (II) or (III) with an HMG-CoA reductase inhibitor (e.g., atorvastatin, fluvastatin, lovastatin, pravastatin, and simvastatin) and aspirin, or a compound of Formula (I), (II) or (III) with an HMG-CoA reductase inhibitor and a β-blocker.

Additionally, an effective amount of a compound of Formula (I), (II) or (III) and a therapeutically effective amount of one or more active agents selected from the group consisting of: an antihyperlipidemic agent; a plasma HDL-raising agent; an antihypercholesterolemic agent, such as a cholesterol biosynthesis inhibitor, for example, an HMG-CoA reductase inhibitor; an HMG-CoA synthase inhibitor; a squalene epoxidase inhibitor, or a squalene synthetase inhibitor (also known as squalene synthase inhibitor); an acyl-coenzyme A cholesterol acyltransferase inhibitor; probucol; nicotinic acid and the salts thereof; CETP inhibitors such as torcetrapib; a cholesterol absorption inhibitor such as ezetimibe; PPAR alpha agonists or partial agonists; PPAR delta agonists or partial agonists; dual PPAR alpha, PPAR delta agonists or partial agonists; dual PPAR alpha, PPAR gamma agonists or partial agonists; dual PPAR delta, PPAR gamma agonists or partial agonists; pan PPAR agonists or partial agonists; niacinamide; a cholesterol absorption inhibitor; a bile acid sequestrant anion exchange resin; a LDL receptor inducer; clofibrate, fenofibrate, and gemfibrozil; vitamin B₆ and the pharmaceutically acceptable salts thereof; vitamin B₁₂; an anti-oxidant vitamin; a β-blocker; an angiotensin II antagonist; an angiotensin converting enzyme inhibitor; a platelet aggregation inhibitor; a fibrinogen receptor antagonist; aspirin; phentiramines, β-3 adrenergic receptor agonists; sulfonylureas, biguanides, α-glucosidase inhibitors, other insulin secretogogues, and insulin can be used together for the preparation of a pharmaceutical composition useful for the above-described treatments.

An additional example of combination therapy can be seen in modulating metabolic syndrome (e.g. treating metabolic syndrome and its related symptoms, complications and disorders), wherein the compounds of Formula (I), (II) or (III) can be effectively used in combination with, for example, the active agents discussed above for modulating or treating diabetes, obesity, hyperlipidemia, atherosclerosis, and/or their respective related symptoms, complications and disorders.

In a further embodiment, a compound of the present invention can be administered in combination with halofenic acid, an ester of halofenic acid, or another prodrug of halofenic acid, preferably with (−)-(4-chlorophenyl)-(3-trifluoromethylphenoxy)-acetic acid 2-acetylaminoethyl ester (MBX-102).

In particular, this invention provides methods of treating a mammal, in particular a human by administering a compound of Formula (I), (II) or (III) and a DPP4 inhibitor.

The DPP4 inhibitors useful in the present invention are sitagliptin (Merck), vildagliptin (Novartis), BMS-477118 (saxagliptin) (Bristol-Myers Squibb), R1438 (amino-methylpyridine) (Roche), NVP DPP728 (Novartis), PSN9301 (Prosidion), P32/98 (isoleucine thiozolidide) (Probiodrug), GSK823093C (Denagliptin) (Glaxo Smithkline), SYR-322 (Alogliptin) (Takeda), N,N-7201 (NovoNordisk), ALS2-0426 (Alantos). (Green B D, Flatt P R, Bailey C J, Dipeptidyl peptidase IB (DPP4) inhibitors: a newly emerging drug class for the treatment of Type II diabetes, Diabetes Vasc. Dis. Res. 2006, 3:159-165). Preferred DPP4 inhibitors are sitagliptin, vildagliptin, Denagliptin, saxagliptin, and alogliptin). Even more preferred CPP4 inhibitors are sitagliptin and vildagliptin.

The compound of Formula (I), (II) or (III) and DPP4 inhibitor are administered in a single dosage or in separate dosages. The single dosage is administered once a day or multiple times a day. When the compound of Formula (I), (II) or (III) and DPP4 inhibitor are administered is separate dosages, the dosages can be administered once a day or multiple times a day.

The dosing of a compound of Formula (I), (II) or (III) and DPP4 inhibitor can be dosed at the same time, within several minutes, or separated by hours. By way of example, a compound of Formula (I), (II) or (III) and DPP4 inhibitor can be dosed together in the morning, with no further dosing for the remainder of the day. Alternatively, in the morning, a compound of Formula (I), (II) or (III) and a DPP4 inhibitor is dosed followed with a second dose of a compound of Formula (I), (II) or (III) and/or a DPP4 inhibitor in the evening or after a meal.

It can be necessary to administer dosages of the compound of Formula (I), (II) or (III) and/or DPP4 inhibitor once a day or more than once a day, or before or after a meal, as will be apparent to those skilled in the art. Further, it is noted that the clinician or treating physician will know how and when to start, interrupt, adjust, or terminate therapy in conjunction with individual patient response.

In one embodiment, when the compound of the present invention and the DPP4 inhibitor are administered in a single dosage, the compound of Formula (I), (II) or (III) and DPP4 inhibitor are formulated into a single pill, single table, or a single capsule. When the compound of Formula (I), (II) or (III) and DPP4 inhibitor are administered in separate dosages, the compound of Formula (I), (II) or (III) is formulated into a pill, tablet or capsule and the DPP4 inhibitor is formulated into a separate pill or capsule.

When the compound of Formula (I), (II) or (III) and DPP4 inhibitor are administered in separate dosages, the compound of this invention can be administered first and the DPP4 inhibitor can be administered next, following administration of the compound of Formula (I), (II) or (III). Alternatively, the DPP4 inhibitor can be administered first and the compound of this invention can be administered next, following administration of the DPP4 inhibitor. The time between the first administration and the second administration can be varied by a skilled practitioner. In one embodiment, the first administration (a compound of Formula (I), (II) or (III) or DPP4 inhibitor), is followed immediately by the second administration (a compound of Formula (I), (II) or (III) or DPP4 inhibitor). In another embodiment, the second administration is within 2 minutes, 5 minutes, 10 minutes, 15 minutes, 30 minutes, or 60 minutes, 1 hour, 2 hours, 3 hours, 4 hours, 5 hours, 6 hours, 7 hours, 8 hours, 9 hours, 10 hours, 11 hours, or 12 hours following the first administration. Yet another embodiment provides for the administration of a compound for Formula (I), (II) or (III) and/or DPP4 inhibitor in the morning followed by the administration of a compound of Formula (I), (II) or (III) and/or DPP4 inhibitor in the evening.

In addition, the present invention provides for kits with unit doses of the compounds of Formula (I), (II) or (III) and/or DPP4 inhibitor, either in oral or injectable doses. In addition to the containers containing the unit doses will be an informational package insert describing the use and attendant benefits of the drugs in treating Type II diabetes, obesity, hyperlipidemia, atherosclerosis and metabolic syndrome, and/or their respective related symptoms, complications and disorders. Preferred compounds and unit doses are those described herein above.

Another aspect of this invention provides methods of lowering blood levels of glucose in a subject by administering a compound of Formula (I), (II) or (III) and a DPP4 inhibitor. The method comprises administering an effective amount of a compound of the invention and DPP4 inhibitor to the mammal. The method further comprises steps to measure blood glucose levels before and after administration of a compound of Formula (I), (II) or (III) and DPP4 inhibitor. Blood glucose levels are easily measured by numerous commercially available glucose monitoring devices that measure blood glucose from samples of blood or urine, or as taught herein. Blood glucose can also be measured by commercially available glucometers that do not require blood or urine samples.

Another aspect of this invention provides methods of lowering blood levels of insulin in a subject by administering a compound of Formula (I), (II) or (III) and a DPP4 inhibitor. The method comprises administering an effective amount of a compound of Formula (I), (II) or (III) and DPP4 inhibitor to the mammal. The method further comprises steps to measure blood insulin levels before and after administration of a compound of this invention and a DPP4 inhibitor. Blood insulin levels are easily measured by well-known insulin monitoring assays that measure insulin from samples of blood or urine, or as taught herein.

In another aspect, this invention provides methods of increasing blood levels of incretins in a subject by administering a compound of this invention and a DPP4 inhibitor. The incretins are GLP-1 and GIP. The method comprises administering an effective amount of a compound of Formula (I), (II) or (III) and DPP4 inhibitor to the mammal. The method further comprises steps to measure blood incretin levels before and after administration of a compound of Formula (I), (II) or (III) and a DPP4 inhibitor. Blood incretin levels are easily measured by well-known incretin monitoring assays, or as taught herein.

Yet another aspect of this invention provides methods of lowering blood triglyceride levels in a subject by administering a compound of Formula (I), (II) or (III) and a DPP4 inhibitor. The method comprises administering an effective amount of a compound of the present invention and DPP4 inhibitor to the mammal. The method further comprises steps to measure blood triglycerides levels before and after administration of a compound of Formula (I), (II) or (III) and DPP4 inhibitor. Blood triglyceride levels are easily measured by numerous commercially available devices that measure blood triglyceride levels from samples of blood.

A further aspect of this invention provides methods of lowering gastric emptying in a subject by administering a compound of the invention and a DPP4 inhibitor. The method comprises administering an effective amount of a compound of Formula (I) and DPP4 inhibitor to the mammal. The method further comprises steps to measure blood incretin levels before and after administration of a compound of Formula (I), (II) or (III) and a DPP4 inhibitor. Blood incretin levels are easily measured by well-known incretin monitoring assays, or as taught herein.

Another aspect of this invention provides methods of increasing insulin production in the islet cells of a subject by administering a compound of Formula (I), (II) or (III) and a DPP4 inhibitor. The method comprises administering an effective amount of a compound of Formula (I), (II) or (III) and DPP4 inhibitor to the mammal. The method further comprises steps to measure insulin production in islet cells or the beta cells of the pancreas before and after administration of a compound of Formula (I), (II) or (III) and a DPP4 inhibitor. The insulin production of islets and beta cells are easily measured by well-known assays, or as taught herein.

In yet another aspect, this invention provides methods of preserving islet function in a subject by administering a compound of the invention and a DPP4 inhibitor. The method comprises administering an effective amount of a compound of Formula (I) and DPP4 inhibitor to the mammal. The method further comprises steps to measure the function of islets or beta cell's ability to produce insulin before and after administration of a compound of Formula (I), (II) or (III) and a DPP4 inhibitor. The insulin production of islets and beta cells are easily measured by well-known assays, or as taught herein.

The compounds of Formula (I), (II) or (III) that are used in the methods of the present invention can be incorporated into a variety of formulations and medicaments for therapeutic administration. More particularly, the compounds of Formula I can be formulated into pharmaceutical compositions by combination with appropriate, pharmaceutically acceptable carriers or diluents, and can be formulated into preparations in solid, semi-solid, liquid or gaseous forms, such as tablets, capsules, pills, powders, granules, dragees, gels, slurries, ointments, solutions, suppositories, injections, inhalants and aerosols. As such, administration of the compounds can be achieved in various ways, including oral, buccal, rectal, parenteral, intraperitoneal, intradermal, transdermal, and/or intratracheal administration. Moreover, the compound can be administered in a local rather than systemic manner, in a depot or sustained release formulation. In addition, the compounds can be administered in a liposome.

The compounds of Formula (I), (II) or (III) can be formulated with common excipients, diluents or carriers, and compressed into tablets, or formulated as elixirs or solutions for convenient oral administration, or administered by the intramuscular or intravenous routes. The compounds can be administered transdermally, and can be formulated as sustained release dosage forms and the like. Compounds of Formula (I), (II) or (III) can be administered alone, in combination with each other, or they can be used in combination with other known compounds.

Suitable formulations for use in the present invention are found in Remington's Pharmaceutical Sciences (Mack Publishing Company (1985) Philadelphia, Pa., 17th ed.), which is incorporated herein by reference. Moreover, for a brief review of methods for drug delivery, see, Langer, Science (1990) 249:1527-1533, which is incorporated herein by reference. The pharmaceutical compositions described herein can be manufactured in a manner that is known to those of skill in the art, i.e., by means of conventional mixing, dissolving, granulating, dragee-making, levigating, emulsifying, encapsulating, entrapping or lyophilizing processes. The following methods and excipients are merely exemplary and are in no way limiting.

For injection, the compound of Formula (I) and DPP4 inhibitor can be formulated into preparations by dissolving, suspending or emulsifying them in an aqueous or nonaqueous solvent, such as vegetable or other similar oils, synthetic aliphatic acid glycerides, esters of higher aliphatic acids or propylene glycol; and if desired, with conventional additives such as solubilizers, isotonic agents, suspending agents, emulsifying agents, stabilizers and preservatives. Preferably, the compounds of the present invention can be formulated in aqueous solutions, preferably in physiologically compatible buffers such as Hanks' solution, Ringer's solution, or physiological saline buffer. For transmucosal administration, penetrants appropriate to the barrier to be permeated are used in the formulation. Such penetrants are generally known in the art.

For oral administration, the compounds of Formula (I) and DPP4 inhibitors can be formulated readily by combining with pharmaceutically acceptable carriers that are well known in the art. Such carriers enable the compounds to be formulated as tablets, pills, dragees, capsules, emulsions, lipophilic and hydrophilic suspensions, liquids, gels, syrups, slurries, suspensions and the like, for oral ingestion by a patient to be treated. Pharmaceutical preparations for oral use can be obtained by mixing the compounds with a solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone. If desired, disintegrating agents can be added, such as the cross-linked polyvinyl pyrrolidone, agar, or alginic acid or a salt thereof such as sodium alginate.

Dragee cores are provided with suitable coatings. For this purpose, concentrated sugar solutions can be used, which can optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments can be added to the tablets or dragee coatings for identification or to characterize different combinations of active compound doses.

Pharmaceutical preparations that can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds can be dissolved or suspended in suitable liquids, such as fatty oils, liquid paraffin, or liquid polyethylene glycols. In addition, stabilizers can be added. All formulations for oral administration should be in dosages suitable for such administration.

For buccal administration, the compositions can take the form of tablets or lozenges formulated in a conventional manner.

For administration by inhalation, the compounds for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas, or from propellant-free, dry-powder inhalers. In the case of a pressurized aerosol the dosage unit can be determined by providing a valve to deliver a metered amount. Capsules and cartridges of, e.g., gelatin for use in an inhaler or insufflator can be formulated containing a powder mix of the compound and a suitable powder base such as lactose or starch.

The compounds can be formulated for parenteral administration by injection, e.g., by bolus injection or continuous infusion. Formulations for injection can be presented in unit dosage form, e.g., in ampoules or in multidose containers, with an added preservative. The compositions can take such forms as suspensions, solutions or emulsions in oily or aqueous vehicles, and can contain formulator agents such as suspending, stabilizing and/or dispersing agents.

Pharmaceutical formulations for parenteral administration include aqueous solutions of the active compounds in water-soluble form. Additionally, suspensions of the active compounds can be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances that increase the viscosity of the suspension, such as sodium carboxymethyl cellulose, sorbitol, or dextran. Optionally, the suspension can also contain suitable stabilizers or agents that increase the solubility of the compounds to allow for the preparation of highly concentrated solutions. Alternatively, the active ingredient can be in powder form for constitution with a suitable vehicle, e.g., sterile pyrogen-free water, before use.

The compounds can also be formulated in rectal compositions such as suppositories or retention enemas, e.g., containing conventional suppository bases such as cocoa butter, carbowaxes, polyethylene glycols or other glycerides, all of which melt at body temperature, yet are solidified at room temperature.

In addition to the formulations described previously, the compounds can also be formulated as a depot preparation. Such long acting formulations can be administered by implantation (for example, subcutaneously or intramuscularly) or by intramuscular injection. Thus, for example, the compounds can be formulated with suitable polymeric or hydrophobic materials (for example, as an emulsion in an acceptable oil) or ion exchange resins, or as sparingly soluble derivatives, for example, as a sparingly soluble salt.

Alternatively, other delivery systems for hydrophobic pharmaceutical compounds can be employed. Liposomes and emulsions are well known examples of delivery vehicles or carriers for hydrophobic drugs. In a presently preferred embodiment, long-circulating, i.e., stealth liposomes can be employed. Such liposomes are generally described in Woodle, et al., U.S. Pat. No. 5,013,556. The compounds of the present invention can also be administered by controlled release means and/or delivery devices such as those described in U.S. Pat. Nos. 3,845,770; 3,916,899; 3,536,809; 3,598,123; and 4,008,719.

Certain organic solvents such as dimethylsulfoxide (“DMSO”) also can be employed, although usually at the cost of greater toxicity. Additionally, the compounds can be delivered using a sustained-release system, such as semipermeable matrices of solid hydrophobic polymers containing the therapeutic agent. Various types of sustained-release materials have been established and are well known by those skilled in the art. Sustained-release capsules can, depending on their chemical nature, release the compounds for a few hours up to over 100 days.

The pharmaceutical compositions also can comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

Pharmaceutical compositions suitable for use in the present invention include compositions wherein the active ingredients are contained in a therapeutically effective amount. The amount of composition administered will, of course, be dependent on the subject being treated, on the subject's weight, the severity of the affliction, the manner of administration and the judgment of the prescribing physician. Determination of an effective amount is well within the capability of those skilled in the art, especially in light of the detailed disclosure provided herein.

For any compound used in the method of the present invention, a therapeutically effective dose can be estimated initially from cell culture assays, animal models, or microdosing of human subjects.

Moreover, toxicity and therapeutic efficacy of the compounds described herein can be determined by standard pharmaceutical procedures in cell cultures or experimental animals, e.g., by determining the LD₅₀, (the dose lethal to 50% of the population) and the ED₅₀ (the dose therapeutically effective in 50% of the population). The dose ratio between toxic and therapeutic effect is the therapeutic index and can be expressed as the ratio between LD₅₀ and ED₅₀. Compounds that exhibit high therapeutic indices are preferred. The data obtained from these cell culture assays and animal studies can be used in formulating a dosage range that is not toxic for use in humans. The dosage of such compounds lies preferably within a range of circulating concentrations that include the ED₅₀ with little or no toxicity. The dosage can vary within this range depending upon the dosage form employed and the route of administration utilized. The exact formulation, route of administration and dosage can be chosen by the individual physician in view of the patient's condition (see, e.g., Fingl, et al., 1975 In: The Pharmacological Basis of Therapeutics, Ch. 1).

The amount of a compound of Formula (I), (II) or (III) that can be combined with a carrier material to produce a single dosage form will vary depending upon the disease treated, the mammalian species, and the particular mode of administration. However, as a general guide, suitable unit doses for the compounds of the present invention can, for example, preferably contain between 0.1 mg to about 1000 mg of the active compound. A preferred unit dose is between 1 mg to about 500 mg. A more preferred unit dose is between 1 mg to about 300 mg. Even more preferred unit dose is between 1 mg to about 100 mg. Such unit doses can be administered more than once a day, for example, 2, 3, 4, 5 or 6 times a day, but preferably 1 or 2 times per day, so that the total dosage for a 70 kg adult is in the range of 0.001 to about 15 mg per kg weight of subject per administration. A preferred dosage is 0.01 to about 1.5 mg per kg weight of subject per administration, and such therapy can extend for a number of weeks or months, and in some cases, years. It will be understood, however, that the specific dose level for any particular patient will depend on a variety of factors including the activity of the specific compound employed; the age, body weight, general health, sex and diet of the individual being treated; the time and route of administration; the rate of excretion; other drugs that have previously been administered; and the severity of the particular disease undergoing therapy, as is well understood by those of skill in the area.

A typical dosage can be one 1 mg to about 100 mg tablet or 1 mg to about 300 mg taken once a day, or, multiple times per day, or one time-release capsule or tablet taken once a day and containing a proportionally higher content of active ingredient. The time-release effect can be obtained by capsule materials that dissolve at different pH values, by capsules that release slowly by osmotic pressure, or by any other known means of controlled release.

It can be necessary to use dosages outside these ranges in some cases as will be apparent to those skilled in the art. Further, it is noted that the clinician or treating physician will know how and when to start, interrupt, adjust, or terminate therapy in conjunction with individual patient response.

For the compositions, methods and kits provided above, one of skill in the art will understand that preferred compounds for use in each are those compounds that are noted as preferred above. Still further preferred compounds for the compositions, methods and kits are those compounds provided in the non-limiting Examples below.

### CHEMICAL EXAMPLES

### Examples

### Experimental Section

General Methods. All operations involving moisture and/or oxygen sensitive materials were conducted under an atmosphere of dry nitrogen in pre-dried glassware. Unless noted otherwise, materials were obtained from commercially available sources and used without further purification.

Flash chromatography was performed on E. Merck silica gel 60 (240-400 mesh) according to the protocol of Still, Kahn, and Mitra (J. Org. Chem. (1978) 43, 2923). Thin layer chromatography was performed using precoated plates purchased from E. Merck (silica gel 60 PF₂₅₄, 0.25 mm) and spots were visualized with long-wave ultraviolet light followed by an appropriate staining reagent.

Nuclear magnetic resonance (“NMR”) spectra were recorded on a Varian Inova-400 resonance spectrometer. ¹H NMR chemical shifts are given in parts per million (δ) downfield from tetramethylsilane (“TMS”) using TMS or the residual solvent signal (CHCl₃=δ 7.24, DMSO=δ 2.50) as internal standard. ¹H NMR information is tabulated in the following format: number of protons, multiplicity (s, singlet; d, doublet; t, triplet; q, quartet; m, multiplet), coupling constant(s) (J) in Hertz, and, in selected cases, position assignment. The prefix app is occasionally applied in cases where the true signal multiplicity was unresolved and br indicates the signal in question was broadened.

The compounds of the present invention can be prepared by methodology in the Reaction Schemes below, and with specific reagents and conditions provided in each of the examples below.

The compounds were named using ChemBioDraw Ultra Version 11.0.

LCMS analysis was performed using a PE SCIEX API 2000 spectrometer with a Phenomenex Luna 5 micron C₁₈ column.

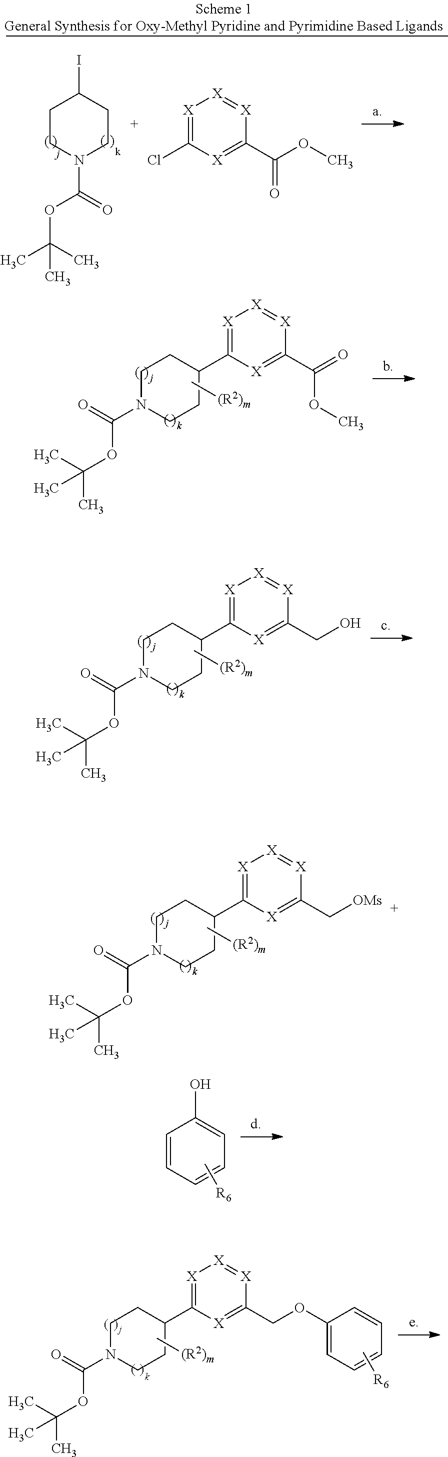

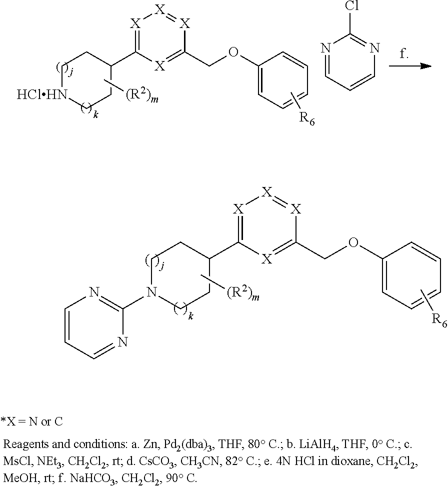

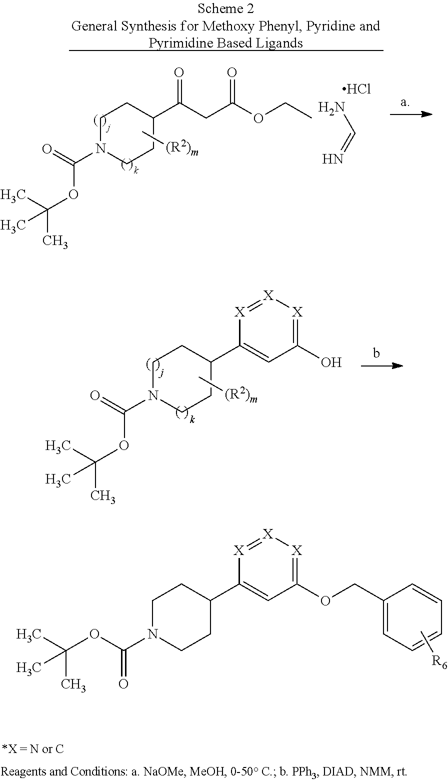

### Example 1

### tert-Butyl 4-(6-(4-(methylsulfonyl)benzyloxy)pyrimidin-4-yl)piperidine-1-carboxylate

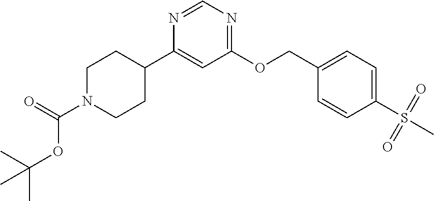

### Step 1: tert-Butyl 4-(6-hydroxypyrimidin-4-yl)piperidine-1-carboxylate

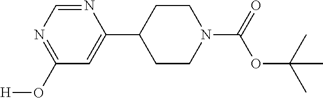

To a solution of 4-(2-ethoxycarbonyl-acetyl)-piperidine-1-carboxylic acid tert-butyl ester (9 g, 30 mmol) in anhydrous methanol (150 mL) was added sodium methoxide (28 mL, 120 mmol) and then formamidine hydrochloride (4.8 g, 60 mmol) at room temperature. The reaction mixture was stirred at room temperature for 70 hours followed by heating at 50° C. for 2 hours. After cooling the room temperature, the mixture was concentrated under in vacuo. The residue was dissolved in water and extracted with diethyl ether. The aqueous phase was acidified with HCl and extracted with CH₂Cl₂. The organic phase was dried over anhydrous sodium sulfate, filtered and concentrated to afford the desired product. ¹H NMR (CDCl₃): δ 8.12 (1H, d), 6.3 (1H, s), 4.25 (2H, br), 2.84 (2H, br), 2.6 (1H, m), 1.88 (2H, m), 1.6 (2H, m), 1.47 (9H, s).

### Step 2: tert-Butyl 4-(6-(4-(methylsulfonyl)benzyloxy)pyrimidin-4-yl)piperidine-1-carboxylate

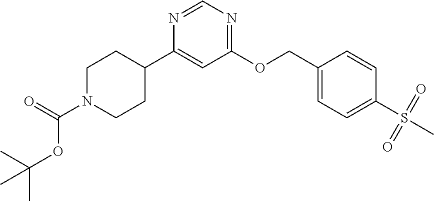

To a solution of tert-butyl 4-(6-hydroxypyrimidin-4-yl)piperidine-1-carboxylate (0.56 g, 2 mmol), (4-methylsulfonyl)benzyl alcohol (0.56 g, 3 mmol) and triphenylphosphine (1.05 g, 4 mmol) in 4-methylmorpholine (10 mL) was added diisopropylazodicarboxylate (0.86 g, 4 mmol) at room temperature. The reaction mixture was stirred at room temperature for 2 hours. The solution was filtered through a pad of celite and the filter cake was washed with ethyl acetate. The filtrate was concentrated and the residue was purified by flash chromatography on silca gel (1:1 hexanes/ethyl acetate) to afford the desired product. ¹H NMR (CDCl₃): δ 8.7 (1H, s), 7.96 (2H, d), 7.64 (2H, d), 6.67 (1H, s), 5.52 (2H, s), 4.25 (2H, br), 3.06 (3H, s), 2.8 (3H, m), 1.95 (2H, m), 1.7 (2H, m), 1.47 (9H, s).

### Example 2

### tert-Butyl 4-(6-((2-fluoro-4-(methylsulfonyl)phenoxy)methyl)pyridin-2-yl)piperidine-1-carboxylate

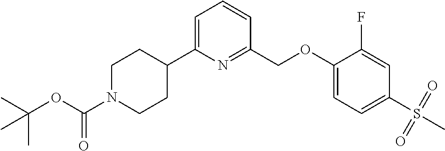

### Step 1: Methyl 6-(1-(tert-butoxycarbonyl)piperidin-4-yl)picolinate

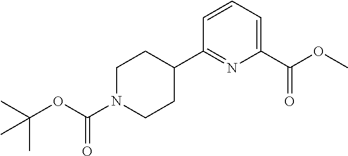

1,2-Dibromoethane (0.15 mL) was added to a suspension of zinc powder (1.3 g) in anhydrous THF (10 mL). The resulting suspension was heated at 65° C. for 5 minutes and then allowed to cool to room temperature. Trimethylsilyl chloride (0.2 mL) was added and the reaction was stirred at room temperature for 30 minutes. N-tert-butoxycarbonyl-4-iodo-piperidine (4.5 g) in THF (10 mL) was added. The reaction mixture was stirred at 50° C. for 2 hours and cooled to room temperature. Meanwhile, a mixture of tri-2-furylphosine (0.2 g) and tris(dibenzylidendacetone)-dipalladium(0) (0.2 g) was dissolved in THF under a nitrogen atmosphere, stirred at room temperature for 30 minutes, and added to the organozinc solution. A solution of methyl 6-chloropicolinate (2.9 g) in THF was added. The reaction mixture was warmed to 80° C. and stirred for 4 hours and subsequently cooled to room temperature, filtered through a pad of celite. The filter cake was washed with ethyl acetate and the filtrate was washed with saturated NaHCO₃, water, and brine. The organic phase was dried over anhydrous sodium sulfate, filtered and concentrated. The residue was purified by flash chromatography on silca gel (1:1 hexanes/ethyl acetate) to afford the desired product. ¹H NMR (CDCl₃): δ 7.97 (1H, d), 7.78 (1H, t), 7.34 (1H, d), 4.26 (2H, br), 3.99 (3H, s), 3.04 (1H, m), 2.84 (2H, m), 1.95 (2H, m), 1.7 (2H, m), 1.47 (9H, s).

### Step 2: tert-Butyl 4-(6-(hydroxymethyl)pyridin-2-yl)piperidine-1-carboxylate

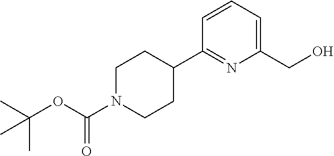

A solution of methyl 6-(1-(tert-butoxycarbonyl)piperidin-4-yl)picolinate (1.6 g, 5 mmol) in THF (25 mL) at 0° C. was treated with LiAlH₄ (0.29 g, 7.5 mmol) and stirred for 1 hour. The reaction mixture was quenched with an aqueous solution of 2 N NaOH. The suspension was filtered through a pad of celite and the filter cake was washed with EtOAc (100 mL). The filtrate was concentrated in vacuo and the residue was purified by flash chromatography on silca gel (1:1 hexanes/ethyl acetate) to afford desired product. ¹H NMR (CDCl₃): δ 7.62 (1H, t), 7.05 (2H, m), 4.72 (2H, d), 4.25 (2H, br), 2.85 (3H, m), 1.9 (2H, m), 1.76 (2H, m), 1.48 (9H, s).

### Step 3: tert-Butyl 4-(6-((methylsulfonyloxy)methyl)pyridin-2-yl)piperidine-1-carboxylate

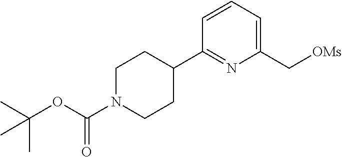

To a solution of tert-butyl 4-(6-(hydroxymethyl)pyridin-2-yl)piperidine-1-carboxylate (1.4 g, 4.8 mmol) in methylene chloride (30 mL) was added triethylamine (0.72 g, 0.72 mmol) and methanesulfonyl chloride (0.66 g, 5.8 mmol) at 0° C. After stirring at 0° C. for 1 hour, the reaction mixture was diluted with EtOAc and washed with H₂O and brine. After drying (Na₂SO₄), the solvent was removed in vacuo. The residue was used in the next step without further purification. ¹H NMR (CDCl₃): δ 7.69 (1H, t), 7.31 (1H, d), 7.15 (1H, d), 5.3 (2H, s), 4.25 (2H, br), 3.08 (3H, s), 2.85 (3H, m), 1.9 (2H, m), 1.7 (2H, m), 1.48 (9H, s).

### Step 4: tert-Butyl 4-(6-((2-fluoro-4-(methylsulfonyl)phenoxy)methyl)pyridin-2-yl)piperidine-1-carboxylate

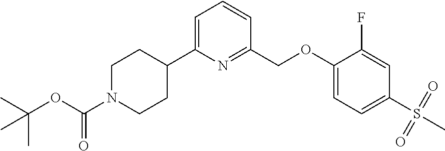

A mixture of tert-butyl 4-(6-((methylsulfonyloxy)methyl)pyridin-2-yl)piperidine-1-carboxylate (0.37 g, 1 mmol), 3-fluoro-4-methanesulfonyl-phenol (0.19 g, 1 mmol) and Cs₂CO₃ (0.65 g, 2 mmol) in acetonitrile (20 mL) was heated to 70° C. for 5 hours. After cooling, the suspension was filtered through a pad of celite. The filtrate was concentrated in vacuo. The crude product was purified by flash chromatography on silica gel (1:1 hexanes/ethyl acetate) to afford the desired product. ¹H NMR (CDCl₃): δ 7.62 (3H, m), 7.31 (1H, d), 7.14 (1H, t), 7.07 (1H, d), 5.25 (2H, s), 4.2 (2H, br), 2.99 (3H, s), 2.8 (3H, m), 1.85 (2H, m), 1.68 (2H, m), 1.43 (9H, s).

### Example 3

### 5-ethyl-2-(4-(6-((2-fluoro-4-(methylsulfonyl)phenoxy)methyl)pyridin-2-yl)piperidin-1-yl)pyrimidine

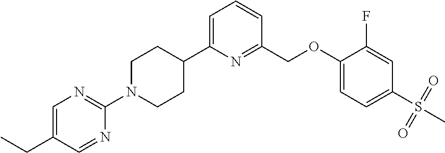

### Step 1: 2-((2-fluoro-4-(methylsulfonyl)phenoxy)methyl)-6-(piperidin-4-yl)pyridine

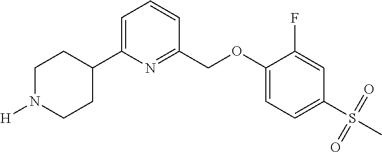

A solution of tert-butyl 4-(6-((2-fluoro-4-(methylsulfonyl)phenoxy)methyl)pyridin-2-yl)piperidine-1-carboxylate (Example 2) in methanol (10 mL) was treated with 10 mL of 4N HCl in dioxane. The resulting solution was stirred at room temperature for 30 minutes. All the solvents were removed in vacuo to afford the desired product as an HCl salt which was used in the next step without further purification.

### Step 2: 5-ethyl-2-(4-(6-((2-fluoro-4-(methylsulfonyl)phenoxy)methyl)pyridin-2-yl)piperidin-1-yl)pyrimidine

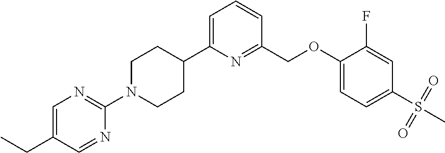

A mixture of 2-((2-fluoro-4-(methylsulfonyl)phenoxy)methyl)-6-(piperidin-4-yl)pyridine (1.0 eq.), 2-chloropyrimidine (1.1 eq.) and K₂CO₃ (4 eq.) in acetonitrile was heated at 82° C. for 4 hours. The suspension was filtered through a pad of celite. The filter cake was washed with ethyl acetate and the solvent was removed in vacuo. The residue was purified on silica gel (1:1 ethyl acetate/hexanes) to afford the desired product. ¹H NMR (CDCl₃): δ 8.2 (2H, s), 7.68 (3H, m), 7.37 (1H, d), 717 (2H, m), 5.31 (2H, s), 4.89 (2H, m), 3.04 (3H, s), 2.99 (3H, m), 2.48 (2H, q), 2.04 (2H, m), 1.79 (2H, m), 1.20 (3H, t).

### Example 4

### tert-Butyl 4-(6-((2-fluoro-4-(1H-tetrazol-1-yl)phenoxy)methyl)pyridin-2-yl)piperidine-1-carboxylate

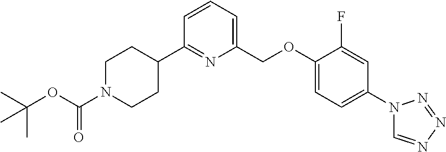

To a solution of tert-butyl 4-(6-((methylsulfonyloxy)methyl)pyridin-2-yl)piperidine-1-carboxylate (Example 2, Step 3) (0.400 g, 1.08 mmol) in acetonitrile (5 mL) were added cesium carbonate (0.528 g, 1.62 mmol), potassium iodide (0.018 g, 0.11 mmol) and 2-fluoro-4-(1H-tetrazol-1-yl)phenol. The solution was heated at 82° C. for 4 hours, cooled to room temperature and filtered through a pad of celite. The filtrate was concentrated under reduced pressure and the resulting film was chromatographed on silica gel (1:1 hexanes/ethyl acetate) to isolate the expected product. ¹H NMR (CDCl₃): δ 8.93 (1H, s), 7.70 (1H, t), 7.54 (1H, dd), 7.39 (2H, m), 7.22 (1H, t), 7.12 (1H, d), 5.31 (2H, s), 4.22 (2H, m), 2.87 (3H, m), 1.97 (2H, m), 1.73 (2H, m), 1.49 (9H, s).

### Example 5

### 5-ethyl-2-(4-(6-((2-fluoro-4-(1H-tetrazol-1-yl)phenoxy)methyl)pyridin-2-yl)piperidin-1-yl)pyrimidine

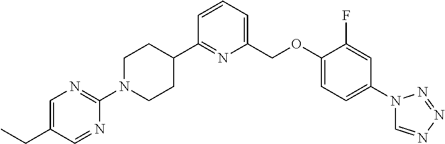

### Step 1: 2-((2-fluoro-4-(1H-tetrazol-1-yl)phenoxy)methyl)-6-(piperidin-4-yl)pyridine hydrochloride

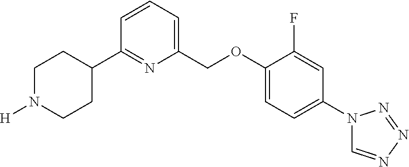

To a solution of tert-butyl 4-(6-((2-fluoro-4-(1H-tetrazol-1-yl)phenoxy)methyl)pyridin-2-yl)piperidine-1-carboxylate (0.200 g, 0.44 mmol) (Example 4) in dichloromethane (2 mL) and methanol (2 mL) was added hydrochloric acid solution (0.6 mL, 4N in dioxane). The solution was stirred for 24 hours at room temperature and was then concentrated to dryness. The white solid was used without further purification.

### Step 2: 5-ethyl-2-(4-(6-((2-fluoro-4-(1H-tetrazol-1-yl)phenoxy)methyl)pyridin-2-yl)piperidin-1-yl)pyrimidine

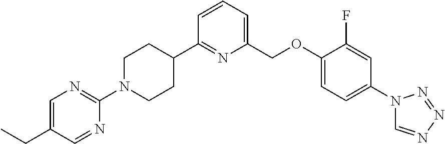

A mixture of 2-((2-fluoro-4-(1H-tetrazol-1-yl)phenoxy)methyl)-6-(piperidin-4-yl)pyridine hydrochloride (0.070 g, 0.179 mmol) (Example 5, Step 1), 2-chloropyrimidine (0.043 mL, 0.358 mmol) and NaHCO₃ (0.075 g, 0.896 mmol) in dimethylformamide (4 mL) was heated at 90° C. for 4 hours. The solution was diluted with water and extracted with ethyl acetate. The organic layer was separated, dried over sodium sulfate, filtered and concentrated in vacuo. The residue was purified on silica gel (1:1 hexanes/ethyl acetate) to afford the desired product. ¹H NMR (CDCl₃): δ 8.91 (1H, s), 8.20 (2H, s), 7.70 (1H, t), 7.53 (1H, dd), 7.39 (2H, m), 7.23 (1H, m), 7.14 (1H, m), 5.31 (2H, s), 4.88 (2H, m), 3.00 (3H, m), 2.49 (2H, q), 2.01 (2H, m), 1.82 (2H, m), 1.20 (3H, t).

### Example 6

### 2-(4-(6-((2-fluoro-4-(1H-tetrazol-1-yl)phenoxy)methyl)pyridin-2-yl)piperidin-1-yl)-5-(trifluoromethyl)pyrimidine

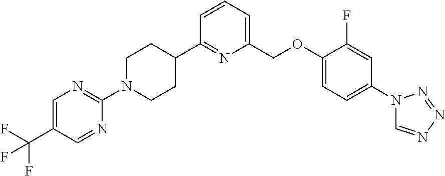

A mixture of 2-((2-fluoro-4-(1H-tetrazol-1-yl)phenoxy)methyl)-6-(piperidin-4-yl)pyridine hydrochloride (0.070 g, 0.179 mmol) (Example 5, Step 1), 2-(methylsulfonyl)-5-(trifluoromethyl)pyrimidine (0.038 g, 0.179 mmol) and NaHCO₃ (0.075 g, 0.896 mmol) in dimethylformamide (4 mL) was stirred for 4 hours at room temperature. The solution was diluted with water and extracted with ethyl acetate. The organic layer was separated, dried over sodium sulfate, filtered and concentrated in vacuo. The residue was purified on silica gel (1:1 hexanes/ethyl acetate) to afford the desired product. ¹H NMR (CDCl₃): δ 8.91 (1H, s), 8.49 (2H, s), 7.70 (1H, t), 7.53 (1H, dd), 7.39 (2H, m), 7.21 (1H, m), 7.13 (1H, m), 5.31 (2H, s), 5.02 (2H, m), 3.07 (3H, m), 2.07 (2H, m), 1.85 (2H, m).

### Example 7

### tert-Butyl 4-(4-((2-fluoro-4-(1H-tetrazol-1-yl)phenoxy)methyl)pyrimidin-2-yl)piperidine-1-carboxylate

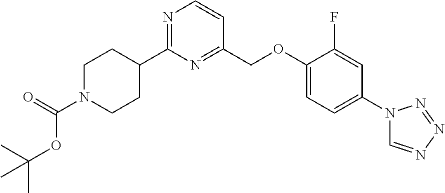

### Step 1: Methyl 2-(1-(tert-butoxycarbonyl)piperidin-4-yl)pyrimidine-4-carboxylate

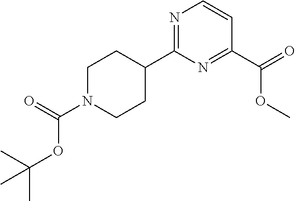

To a slurry of Rieke zinc (0.252 g, 3.86 mmol) in THF (5 mL) was added tert-butyl 4-iodopiperidine-1-carboxylate (1.06 g, 3.21 mmol). The suspension was stirred at 50° C. for 1.5 hours and then cooled to room temperature. Meanwhile, in a separate flask, a mixture of tri-2-furylphosine (0.060 g, 0.256 mmol) and tris(dibenzylidendacetone)-dipalladium(0) (0.066 g, 0.064 mmol) was stirred in THF under a nitrogen atmosphere for 30 minutes. The contents were subsequently added to the organozinc solution. Methyl 2-chloropyrimidine-4-carboxylate (0.72 g, 4.17 mmol) (see US PCT 2007/225271 A1 ex. C4.1) in a solution of THF (5 mL) and DMF (2 mL) was immediately added to the mixture. The solution was then stirred at 80° C. for 3.5 hours. After cooling to room temperature, the solution was diluted with water and extracted with ethyl acetate. The organic layer was separated, dried over sodium sulfate, filtered and concentrated in vacuo. The residue was purified on silica gel (1:1 hexanes/ethyl acetate) to afford the desired product. ¹H NMR (CDCl₃): δ 8.91 (1H, d), 7.80 (1H, d), 4.21 (2H, m), 4.01 (3H, s), 3.18 (1H, m), 2.87 (2H, m), 1.99 (2H, m), 1.84 (2H, m), 1.43 (9H, s).

### Step 2: tert-Butyl 4-(4-(hydroxymethyl)pyrimidin-2-yl)piperidine-1-carboxylate

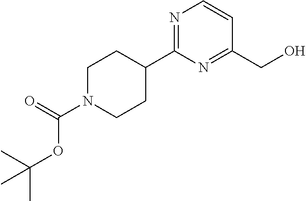

A solution of methyl 2-(1-(tert-butoxycarbonyl)piperidin-4-yl)pyrimidine-4-carboxylate (0.155 g, 0.483 mmol) in THF (5 mL) at 0° C. was treated with LiAlH₄ (0.022 g, 0.519 mmol). The mixture was stirred for 1 hour. The reaction mixture was quenched with a 2 normal aqueous NaOH solution. The slurry was filtered through a pad of celite and the filter cake was washed with EtOAc. The organic layer was separated, dried over sodium sulfate, filtered and concentrated under reduced pressure. The residue was purified by flash chromatography on silica gel (1:2 hexanes/ethyl acetate) to afford the desired product. ¹H NMR (CDCl₃): δ 8.61 (1H, d), 7.16 (1H, d), 4.75 (2H, s), 4.21 (2H, m), 3.08 (1H, m), 2.88 (2H, m), 1.82 (2H, m), 1.84 (2H, m), 1.43 (9H, s).

### Step 3: tert-Butyl 4-(4-((methylsulfonyloxy)methyl)pyrimidin-2-yl)piperidine-1-carboxylate

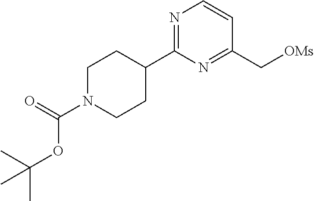

Tert-butyl 4-(4-((methylsulfonyloxy)methyl)pyrimidin-2-yl)piperidine-1-carboxylate was synthesized from tert-butyl 4-(4-(hydroxymethyl)pyrimidin-2-yl)piperidine-1-carboxylate (Example 7, Step 2) in a similar manner as described in Example 2, Step 3. This compound was used in the next step without further purification.

### Step 4: tert-Butyl 4-(4-((2-fluoro-4-(1H-tetrazol-1-yl)phenoxy)methyl)pyrimidin-2-yl)piperidine-1-carboxylate

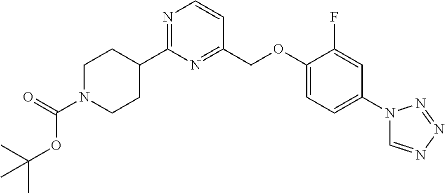

Tert-butyl 4-(4-((2-fluoro-4-(1H-tetrazol-1-yl)phenoxy)methyl)pyrimidin-2-yl)piperidine-1-carboxylate was synthesized from tert-butyl 4-(4-((methylsulfonyloxy)methyl)pyrimidin-2-yl)piperidine-1-carboxylate and 2-fluoro-4-(1H-tetrazol-1-yl)phenol (Example 7, Step 3) in a similar manner as described in Example 2, Step 4. ¹H NMR (CDCl₃): δ 8.98 (1H, s), 8.65 (1H, d), 7.66 (1H, d), 7.39 (2H, m), 7.17 (1H, m), 5.21 (2H, d), 4.19 (2H, m), 2.99 (1H, m), 2.78 (2H, m), 1.98 (2H, m), 1.77 (2H, m), 1.41 (9H, s).

### Example 8

### 2-(1-(5-chloropyrimidin-2-yl)piperidin-4-yl)-4-((2-fluoro-4-(1H-tetrazol-1-yl)phenoxy)methyl)pyrimidine

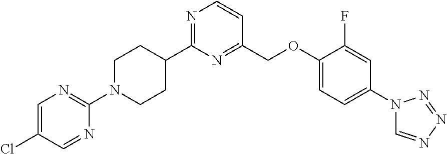

### Step 1: 4-((2-fluoro-4-(1H-tetrazol-1-yl)phenoxy)methyl)-2-(piperidin-4-yl)pyrimidine hydrochloride

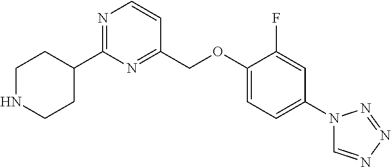

4-((2-fluoro-4-(1H-tetrazol-1-yl)phenoxy)methyl)-2-(piperidin-4-yl)pyrimidine hydrochloride was synthesized from tert-butyl 4-(4-((2-fluoro-4-(1H-tetrazol-1-yl)phenoxy)methyl)pyrimidin-2-yl)piperidine-1-carboxylate (Example 7) and a solution of hydrochloric acid in dioxane in a similar manner as described in Example 5, Step 1. The product was used in the next step without further purification.

### Step 2: 2-(1-(5-chloropyrimidin-2-yl)piperidin-4-yl)-4-((2-fluoro-4-(1H-tetrazol-1-yl)phenoxy)methyl)pyrimidine

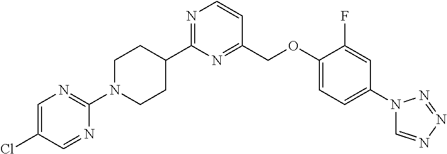

2-(1-(5-chloropyrimidin-2-yl)piperidin-4-yl)-4-((2-fluoro-4-(1H-tetrazol-1-yl)phenoxy)methyl)pyrimidine was synthesized from 4-((2-fluoro-4-(1H-tetrazol-1-yl)phenoxy)methyl)-2-(piperidin-4-yl)pyrimidine hydrochloride (Example 8, Step 1) and 5-chloro-2-iodopyrimidine in a similar manner as described in Example 5, Step 2. ¹H NMR (CDCl₃): δ 8.87 (1H, s), 8.68 (1H, d), 8.16 (2H, s), 7.50 (1H, dd), 7.38 (2H, m), 7.11 (1H, m), 5.19 (2H, s), 4.75 (2H, m), 3.11 (1H, m), 3.00 (2H, m), 2.02 (2H, m), 1.84 (2H, m).

### Example 9

tert-Butyl 4-(3-(4-(methylsulfonyl)benzyloxy)phenyl)piperidine-1-carboxylate

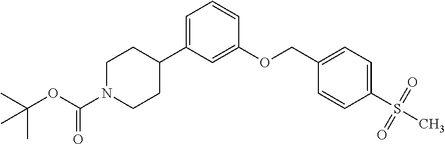

### Step 1: tert-Butyl 4-(3-hydroxyphenyl)piperidine-1-carboxylate

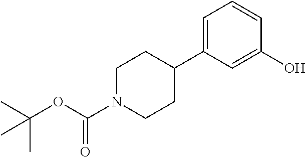

Tert-butyl 4-(3-hydroxyphenyl)piperidine-1-carboxylate was synthesized from 3-(piperidin-4-yl)phenol in a similar manner as described in Syn. Lett. Vol. 5, 2007, 806-808.

### Step 2: tert-Butyl 4-(3-(4-(methylsulfonyl)benzyloxy)phenyl)piperidine-1-carboxylate

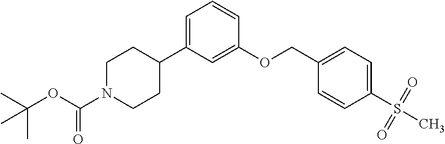

Tert-butyl 4-(3-(4-(methylsulfonyl)benzyloxy)phenyl)piperidine-1-carboxylate was synthesized from tert-butyl 4-(3-hydroxyphenyl)piperidine-1-carboxylate and 1-(chloromethyl)-4-(methylsulfonyl)benzene in a similar manner as described in Example 2, Step 4. ¹H NMR (CDCl₃): δ 7.97 (2H, d), 7.65 (2H, d), 7.23 (1H, m), 6.81 (3H, m), 5.15 (2H, s), 4.25 (2H, m), 3.04 (3H, s), 2.79 (2H, m), 2.62 (1H, m), 1.82 (2H, m), 1.60 (2H, m), 1.48 (9H, s).

### Example 10

### 5-ethyl-2-(4-(3-(4-(methylsulfonyl)benzyloxy)phenyl)piperidin-1-yl)pyrimidine

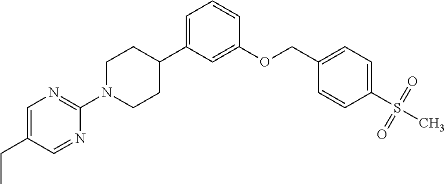

### Step 1: 4-(3-(4-(methylsulfonyl)benzyloxy)phenyl)piperidine hydrochloride

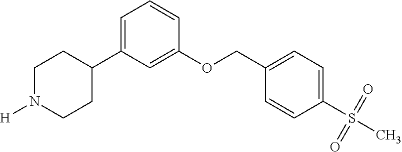

4-(3-(4-(methylsulfonyl)benzyloxy)phenyl)piperidine hydrochloride was synthesized from tert-butyl 4-(3-(4-(methylsulfonyl)benzyloxy)phenyl)piperidine-1-carboxylate (Example 9) and a solution of hydrochloric acid in dioxane in a similar manner as described in example Example 5, Step 1. The product was used in the next step without further purification.

### Step 2: 5-ethyl-2-(4-(3-(4-(methylsulfonyl)benzyloxy)phenyl)piperidin-1-yl)pyrimidine

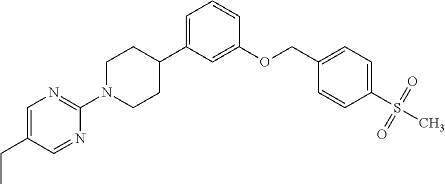

5-ethyl-2-(4-(3-(4-(methylsulfonyl)benzyloxy)phenyl)piperidin-1-yl)pyrimidine was synthesized from 4-(3-(4-(methylsulfonyl)benzyloxy)phenyl)piperidine hydrochloride and 2-chloro-5-ethylpyrimidine in a similar manner as described in Example 5, Step 2. ¹H NMR (CDCl₃): δ 8.17 (2H, s), 7.94 (2H, d), 7.62 (2H, d), 7.22 (1H, m), 6.84 (2H, m), 6.78 (1H, m), 5.12 (2H, s), 4.68 (2H, m), 3.04 (3H, s), 2.93 (2H, m), 2.75 (1H, m), 2.45 (2H, m), 1.90 (2H, m), 1.67 (2H, m), 1.18 (3H, t).

### Example 11

### tert-Butyl 4-(3-(4-(methylsulfonyl)benzylamino)methyl)pyridin-2-yl)piperidine-1-carboxylate

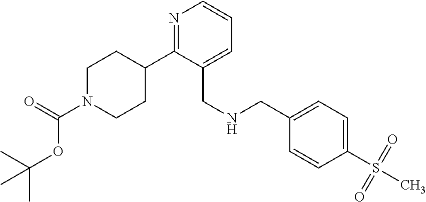

Tert-butyl 4-(3-(aminomethyl)pyridine-2-yl)piperidine-1-carboxylate (300 mg, 1.03 mmol) was dissolved in N-methylpyrrolidinone (5 mL), and diisopropylethylamine (3 eq.) was added to the reaction followed by 4-methylsulfonylbenzylbromide (1.5 eq.). The reaction mixture was heated and stirred at 115° C. for 3 hrs. Upon completion, the reaction was quenched with water and extracted with ethyl acetate. The organic layer was washed with brine, dried over sodium sulfate, filtered and concentrated under reduced pressure. The residue was purified by preparative HPLC. ¹H NMR (CDCl₃): δ 8.54 (1H, br), 8.47 (1H, d), 7.85 (2H, d), 7.63 (1H, d), 7.52 (2H, d), 7.22 (1H, dd), 4.18 (2H, m), 3.72 (2H, s), 3.65 (2H, s), 3.07 (1H, m), 3.04 (3H, s), 2.61 (2H, m), 1.82 (2H, m), 1.56 (2H, m), 1.45 (9H, s).

### Example 12

### tert-Butyl 4-(5-((4-(1H-tetrazol-1-yl)phenoxy)methyl)pyridin-2-yl)piperidine-1-carboxylate

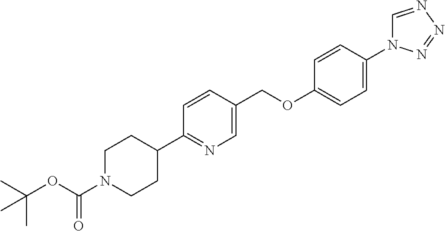

To a solution of tert-butyl 4-(5-(hydroxymethyl)pyridine-2-yl)piperidine-1-carboxylate (250 mg, 0.86 mmol), 4-(1H-tetrazol-1-yl)phenol (1.5 eq.) and triphenylphosphine (1.5 eq.) in THF (7 mL) at 0° C. was added a solution of di-tert-butyl azodicarboxylate (1.5 eq.) in THF (3 mL). After the addition was complete, the reaction was allowed to stir at 0° C. for 1 hr and then at room temperature for an additional 16 hrs. After removal of the solvent in vacuo, the residue. The residue was purified by flash chromatography on silica gel (1:2 hexanes/ethyl acetate) to afford the desired product as a white solid. ¹H NMR (CDCl₃): δ 8.91 (1H, s), 8.62 (1H, s), 7.44-7.76 (4H, m), 7.22 (1H, m), 7.14 (1H, m), 5.12 (2H, s), 4.27 (2H, m), 2.88 (3H, m), 1.93 (2H, m), 1.72 (2H, m), 1.48 (9H, s).

### Example 13

### (4-(6-((2-fluoro-4-(methylsulfonyl)phenoxy)methyl)pyridin-2-yl)piperidin-1-yl)(3-isopropyl-1,2,4-oxadiazol-5-yl)methanone

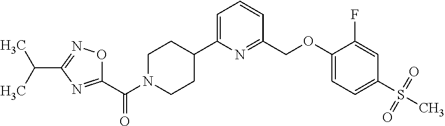

2-((2-fluoro-4-(methylsulfonyl)phenoxy)methyl)-6-(piperidin-4-yl)pyridine (Example 3, Step 1) was synthesized using 3-isopropyl-5-(trichloromethyl)-1,2,4-oxadiazole in a manner similar to that described in example 10, step 2. ¹H NMR (CDCl₃): δ 7.66 (3H, m), 7.38 (1H, d), 7.17 (1H, d), 7.12 (1H, m), 5.29 (2H, s), 4.8 (1H, m), 4.18 (1H, m), 3.3 (1H, m), 3.6 (1H, m), 3.02 (3H, s), 3.0 (2H, m), 2.05 (2H, m), 1.9 (2H, m), 1.35 (6H, d).

### Example 14

### tert-Butyl 4-(4-((2-fluoro-4-(1H-tetrazol-1-yl)phenoxy)methyl)pyridin-2-yl)piperidine-1-carboxylate

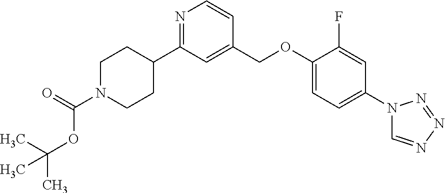

The title compound was obtained in a manner similar to that described in Example 2 using methyl-2-chloroisonicotinate as the starting material.

### Biological Example 1

### Stimulation of cAMP

Generation of GPR119 Stable Cell Line

The compounds of the present invention were evaluated in an assay demonstrating agonism of GPR119. This assay was developed using a stable cell line expressing GPR119, generated as follows. GPR119 (co-pending, co-owned patent application U.S. Ser. No. 11/964,461) was cloned into Gateway pDEST 40vector (Invitrogen), using the Gateway cloning system (invitrogen) according to the manufacturer's instructions. A stable cell line was generated by transfecting a 10 cm plate of CHO cells (source) with Bug of this construct using Transit-CHO transfection kit (Minis). CHO cells were plated the day prior to transfection at a density of 3,000,000 cells/plate. Clones were selected using the antibiotic G418 at 500 ug/ml. 23 clones were picked and assayed for the expression of the receptor by measuring changes in intracellular cAMP levels in response to a known GPR119 agonist.

To measure cAMP activity in response to GPR119 agonist, the clones were plated in 96 well plates at 17500 cells per well. On the day after plating, cells were incubated with the GPR119 agonist at 10 μM for 30 minutes in Ham's F12 Media (Gibco) with 0.04% DMSO. cAMP was measured using the cAMP dynamic kit from Cis Bio (Bedford, Mass.) according to the manufacturer's instructions. Briefly, cells were lysed, and cAMP levels determined by competitive immunoassay using D2 labeled cAMP, and europium cryptate tagged anti cAMP antibody. When in close proximity, the D2 and europium cryptate undergo fluorescence resonance energy transfer (FRET), which is measured as a fluorescence ratio (665 nm/620 nm). Unlabelled cAMP in the cell lysate competed with the D2 labeled cAMP for the europium crypate labeled antibody. The resulting decrease in FRET signal corresponded to intracellular cAMP levels. Fluorescence was read on a BMG Labtech PHERAstar, software version 1.50.

The clone with the greatest response to GPR119 agonist was selected for the screening assay.

Determination of Activity of Compounds

Compounds were dissolved in 100% DMSO to a concentration of 10 μM to provide stock solutions. To determine activity against GPR119, compounds were incubated with GPR119 stably expressing cells (described above), at 6-8 concentrations ranging from 0.00003 to 10 micromolar, in 96 well plates, in 50 ul of Ham's F12 media for 30 minutes. Cells were plated at 17500 cells per well 1 day before running the assay. All compounds were also screened against the parental CHO cells. cAMP was measured using the cAMP dynamic kit from Cis Bio (Bedford, Mass.), according to the manufacturer's instructions. Briefly, cells were lysed and cAMP levels determined by competitive immunoassay using D2 labeled cAMP, and europium cryptate tagged anti cAMP antibody. When in close proximity, the D2 and europium cryptate undergo fluorescence resonance energy transfer (FRET), which is measured as a fluorescence ratio (665 nm/620 nm). Unlabelled cAMP in the cell lysate competed with the D2 labeled cAMP for the europium crypate labeled antibody. The resulting decrease in FRET signal corresponded to intracellular cAMP levels.

To determine percent activity for a tested compound, the FRET signal value obtained at a particular concentration are compared to the Maximal FRET signal value obtained for 5-Ethyl-2-{4-[4-(4-tetrazol-1-yl-phenoxymethyl)-thiazol-2-yl]-piperidin-1-yl}-pyrimidine. The maximal activity of 5-Ethyl-2-{4-[4-(4-tetrazol-1-yl-phenoxymethyl)-thiazol-2-yl]-piperidin-1-yl}-pyrimidine is designated as 100% activity. Typically, the concentration of 5-Ethyl-2-{4-[4-(4-tetrazol-1-yl-phenoxymethyl)-thiazol-2-yl]-piperidin-1-yl}-pyrimidine in the assay was approximately 0.1 μM. The synthesis of 5-Ethyl-2-{4-[4-(4-tetrazol-1-yl-phenoxymethyl)-thiazol-2-yl]-piperidin-1-yl}-pyrimidine is disclosed in co-owned pending U.S. patent application Ser. No. 11/964,461, herein incorporated by reference. Activities of compounds in Table 1 below are expressed as % activity at 3 μM compound compared to the maximal activity of 5-Ethyl-2-{4-[4-(4-tetrazol-1-yl-phenoxymethyl)-thiazol-2-yl]-piperidin-1-yl}-pyrimidine at 3 μM.

| | | % Activity at 3 μM |
|---|---|---|
| Compound | Compound Structures | Compound |
| Example 1 | 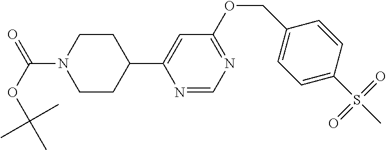 | Not Tested |
| Example 2 | 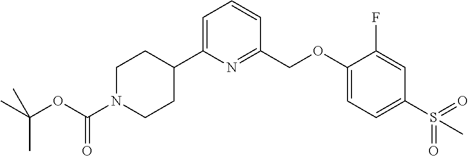 | 59 |
| Example 3 | 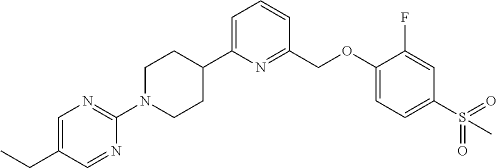 | Not Tested |
| Example 4 | 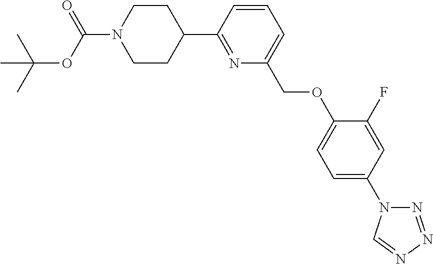 | 61 |
| Example 5 | 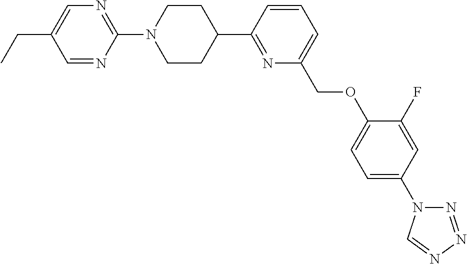 | 73 |
| Example 6 | 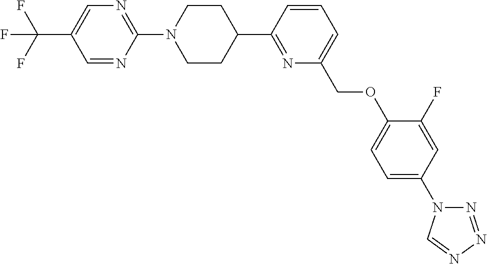 | 63 |
| Example 7 | 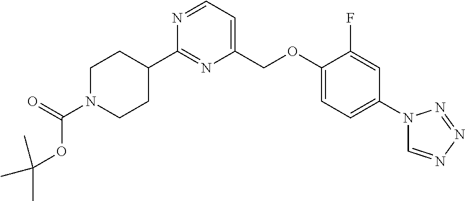 | 70 |
| Example 8 | 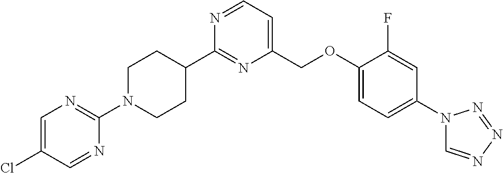 | 72 |
| Example 9 | 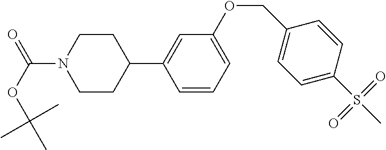 | 61 |
| Example 10 | 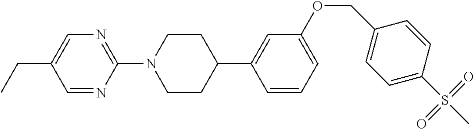 | 59 |
| Example 13 | 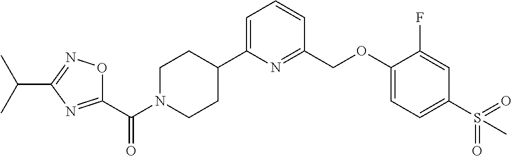 | 33 |
| Example 14 | 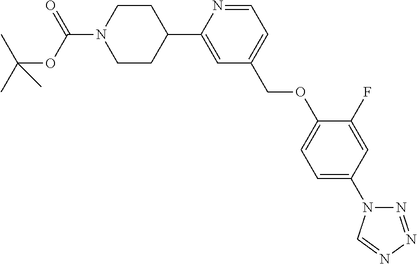 | 73 |

### Biological Example 2

### Insulin Secretion (Islet Perifusion)

To determine the effect of GPR119 agonists on insulin secretion from islets, islets from Sprague Dawley rats are isolated. 200-250 g Sprague Dawley rats (Charles River laboratories) are maintained on regular chow (Purina 5001). Before the procedure rats are anesthetized with intra peritoneal injection of pentobarbital at 200 mg/kg. The bile duct is clamped where it enters the duodenum, then a catheter is placed in the bile duct between the liver and the pancreas. The pancreas is infused through the catheter with a solution of 0.75 mg/ml collagenase P (Roche) in HBSS buffer (Biowhitaker) supplemented with 0.1% glucose and 0.02% BSA. The pancreas is then excised from the rat and placed in 5 ml of the collagenase P solution in a 37° C. waterbath for 8 minutes. After 8 minutes the digested pancreas is shaken vigorously by hand for 30 seconds. The resulting digest is washed four times in the HBSS buffer, then applied to a discontinuous ficoll gradient. To make the gradient, the digest is resuspended in 7.5 ml of ficoll DL400 solution (Sigma) density 1.108, in a 15 ml tube. Three 2 ml layers of ficoll solution of decreasing density (1.096, 1.069, 1.037) are then added to the tube to create a density gradient. The gradient is centrifuged at 1500 rpm for 15 minutes after which islets are picked from the top two layers. Islets are washed four times in HBSS buffer, then cultured in RPMI 1640 media (Gibco) supplemented with 1% fetal bovine serum. The following day, 25 size-matched islets are placed in a perifusion chamber and exposed to Krebs Ringer Buffer (KRB; 119 mM NaCl, 4.7 mM KCl, 25 mM NaHCO₃, 2.5 mM CaCl₂, 1.2 mM MgSO₄, 1.2 mM KH2PO₄) at a rate of 1 ml/minute, using a Cellex Acu-sys S perifusion culture system. The islets are exposed to KRB containing glucose at 2 mM for 30 minutes, followed with buffer containing 16 mM glucose for 30 minutes, then returned to 2 mM glucose for a further 30 minutes, in the presence of 0.1-100 μM of the GPR119 agonist or vehicle (DMSO). Perifusate is collected at 1 minute intervals using a fraction collector, and assayed for insulin using an ELISA kit (Mercodia Ultrasensitive Rat Insulin ELISA Kit, ALPCO). Insulin secretion rate in response to glucose is plotted against time, and the AUC of the curve determined in order to quantify the insulin secretory response to 16 mM glucose during the 30 minute perifusion. Statistical significance of differences in AUC between treated and untreated islets are determined by paired Students t test.

### Biological Example 3

### Oral Glucose Tolerance

8-10 week old male C57/6J mice (Harlan) are maintained on regular chow diet (Purina 5001). The day of the experiment mice are fasted for 6 hours, then randomized into groups (n=8) to receive the tested GPR119 agonist at doses ranging from 0.3-30 mg/kg or the vehicle (1% CMC, 2% TWEEN 80). Compounds are delivered orally via gavage at ml/kg. Blood glucose levels are measured by glucometer (Ascensia Elite XL, Bayer) at time 0, before administration of compound. Blood glucose is measured again after 30 minutes, and then the mice are dosed orally with 2 g/kg glucose at 10 ml/kg. Blood glucose measurements are taken 15, 30, 60, 90 and 120 minutes after glucose administration by glucometer (Ascensia Elite XL, Bayer).

Glucose levels are plotted against time, and the incremental area under the curve (AUC) of the glucose excursion are determined from time 0 using Graphpad Prism 5.0. Outliers are excluded using Tukey's box plot outlier test, and statistical significance of differences in AUC of compound treatment compared to vehicle are determined by non-parametric Kruskal-Wallis test with Dunn's post test.

### Biological Example 4

### Incretin Measurement

The effect of GPR119 agonists on the secretion of insulin, Glucagon-like peptide-1 (GLP-1) and GIP in C57/6J mice are determined as follows.

8-10 week old male C57/6J mice (Harlan) are maintained on a regular chow diet (Purina 5001). On the day of the experiment mice are fasted for 6 hours then randomized into groups (n=8). All groups are treated with the DPPIV inhibitor sitagliptin at 100 mg/kg to prevent degredation of active GLP-1. IC-GPCR-2 agonist compounds are dosed at concentrations ranging from 0.3-300 mg/kg in 1% CMC, 2% TWEEN 80 at −30 minutes. Sitagliptin is administered in the same dosing solution. Oral glucose at 2 g/kg is adminsted at 0 minutes. At 10 minutes after glucose administration, animals are anesthetized with pentobarbital (40 mg/ml in 10% ethanol) and blood collected by heart puncture in microtainer tubes (BD) with potassium EDTA. For GLP-1 assay, the collection tubes also contain a DPP-IV inhibitor provided in the GLP-1 assay kit.

Insulin is measured using the Mercodia mouse Insulin ELISA Kit (ALPCO) according to the manufacturer's instructions. Bioactive GLP-1 is measured using Glucagon-like peptide-1 (active) ELISA assay kit (Linco) according to the manufacturer's instructions. GIP is measured using rat/mouse GIP total ELISA assay kit (Linco), according to the manufacturer's instructions.

### Biological Example 5

### Improvement of Diabetes Parameters in Female ZDF Rat

Female ZDF rats (Charles River laboratories) are obtained at 6 weeks of age and acclimatized for 1 week before being placed on a high fat diet (RD 13004, Research Diets). Compounds are administered to the rats by daily gavage at concentrations ranging from 0.3-300 mg/kg in 1% CMC, 2% TWEEN 80. Body weight and food intake is monitored daily. After 14 days of dosing, blood samples are taken from overnight fasted animals to measure glucose and insulin. Glucose is measured using a glucometer (Ascensia Elite XL, Bayer), insulin is measured using rat insulin ELISA kit (ALPCO). Insulin and glucose levels are compared to those of vehicle treated animals to determine efficacy.

All patents, patent applications, publications and presentations referred to herein are incorporated by reference in their entirety. Any conflict between any reference cited herein and the teaching of this specification is to be resolved in favor of the latter. Similarly, any conflict between an art-recognized definition of a word or phrase and a definition of the word or phrase as provided in this specification is to be resolved in favor of the latter.

1. A compound having the Formula (I): 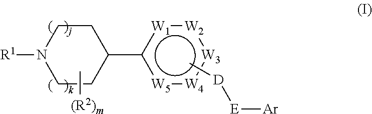
wherein;
W₁, W₂, W₃, W₄ and W₅ are independently selected from the group consisting of CR³, and N, provided that only zero, one, two, or three of W₁, W₂, W₃, W₄ and W₅ is N;
D, and E are independently selected from the group consisting of a bond, —(CHR⁴)ₚ—, —C(O)—, —O—, —S—, —S(O)—, —S(O)₂—, and —NR⁵—, provided that one of D or E is —(CHR⁴)ₚ— or —C(O)— and wherein;
p is 0, 1, or 2;
j is 0, 1, or 2;
k is 0, 1, or 2;
m is 0, 1, 2, 3, or 4;
Ar is a 5- to 10-membered aryl or heteroaryl group, optionally substituted with from one to five R⁶ groups;
R¹ is selected from the group consisting of H, C₁₋₁₀alkyl, C₁₋₁₀substituted alkyl, C₃₋₇cycloalkyl, C₂₋₁₀alkenyl, C₂₋₁₀alkynyl, —X¹—COR^{a}, —X¹—CO₂R^{a}, —X¹—CONR^{a}R^{b}, —SO₂R^{a}, a 4- to 7-membered heterocyclyl group, aryl and a 5- to 10-membered heteroaryl group, wherein each of said cycloalkyl group, heterocyclyl group, aryl group and heteroaryl group is optionally substituted with from 1 to 4 substituents independently selected from the group consisting of halo, C₁₋₁₀alkyl, C₁₋₁₀-substituted alkyl, C₃₋₇cycloalkyl, C₂₋₁₀alkenyl, C₂₋₁₀alkynyl, aryl, heteroaryl, —CN,
—NR^{a}COR^{b}, —NR^{a}CONR^{a}R^{b}, —NO₂, —OR^{a}, —NR^{a}R^{b}, —COR^{a}, —CO₂R^{a}, —CONR^{a}R^{b}, —SR^{a}, —S(O)R^{a}, —S(O)₂R^{a}, —NR^{a}S(O)₂R^{b}, and —SO₂NR^{a}R^{b}, or optionally R^{a} and R^{b} are combined to form a 4-, 5- or 6-membered ring, and X¹ is selected from the group consisting of a bond, C₁₋₄alkylene, C₂₋₆alkenylene, C₂₋₆alkynylene, —C(O)—, and —C(O)—(CH₂)₁₋₄—, wherein the aliphatic portions of X¹ are optionally substituted with one to three members selected from the group consisting of halo, C₁₋₄alkyl, C₁₋₄substituted alkyl and C₁₋₄haloalkyl;
each R² is independently selected from the group consisting of H, halo, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b}, and wherein when the subscript m is 2 and R² is alkyl or substituted alkyl, the two R² members can optionally cyclize to form a ring;
R³ is selected from the group consisting of H, halo, cyano, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b};
each R⁴ is independently selected from the group consisting of H, halo, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b};
R⁵ is selected from the group consisting of H, C₁₋₅alkyl, and C₁₋₅substituted alkyl;
each R⁶ is independently selected from the group consisting of H, halo, C₁₋₁₀alkyl, C₁₋₁₀substituted alkyl, C₃₋₇cycloalkyl, C₂₋₁₀alkenyl, C₂₋₁₀alkynyl, CN, NO₂, —OR^{a}, —NR^{a}R^{b}, —COR^{a}, —CO₂R^{a}, —CONR^{a}R^{b}, —NR^{a}COR^{b}, —NR^{a}CO₂R^{b}, —NR^{a}CONR^{a}R^{b}, —SR^{a}, —S(O)R^{a}, —S(O)₂R^{a}, —NR^{a}S(O)R^{b}, —NR^{a}S(O)₂R^{b}, —SO₂NR^{a}R^{b}, a 4- to 7-membered heterocyclyl group, aryl and a 5- to 10-membered heteroaryl group, wherein each of said heterocyclyl groups, said aryl and heteroaryl groups are optionally substituted with from one to four substituents independently selected from the group consisting of halo, oxo, C₁₋₄alkyl, C₁₋₄haloalkyl, C₃₋₇cycloalkyl, CN, NO₂, —OR^{a}, —NR^{a}R^{b}, —COR^{a}, —CO₂R^{a}, —CONR^{a}R^{b}, —NR^{a}COR^{b}, —NR^{a}CO₂R^{b},
—NR^{a}CONR^{a}R^{b}, —SR^{a}, —S(O)R^{a}, —S(O)₂R^{a}, —NR^{a}SO₂R^{b}, and —SO₂NR^{a}R^{b}, and optionally R^{a} and R^{b} are combined to form a 4-, 5- or 6-membered ring;
and each R^{a} and R^{b} is independently selected from the group consisting of hydrogen, C₁₋₁₀alkyl, C₁₋₁₀haloalkyl, C₃₋₁₀cycloalkyl, heterocyclyl, C₂₋₁₀alkenyl, C₂₋₁₀alkynyl, aryl, substituted aryl, 5- to 6-membered heteroaryl, 5- to 6-membered substituted heteroaryl, and arylC₁₋₄alkyl; and wherein the aliphatic portions of each of said R^{a} and R^{b} is optionally substituted with from one to three members selected from the group consisting of halo, —ORⁿ, —OCORⁿ, —OC(O)N(Rⁿ)₂, —SRⁿ, —S(O)Rⁿ, —S(O)₂Rⁿ,
—S(O)₂N(Rⁿ)₂, —NRⁿS(O)₂Rⁿ, —C(O)N(Rⁿ)₂, —C(O)Rⁿ, —NRⁿC(O)Rⁿ, —NRⁿC(O)N(Rⁿ)₂, —CO₂Rⁿ, —NRⁿCO₂Rⁿ, —CN, —NO₂, —N(Rⁿ)₂ and —NRⁿS(O)₂N(Rⁿ)₂, wherein each Rⁿ is independently hydrogen or an unsubstituted C₁₋₆alkyl;
or pharmaceutically acceptable salts, solvates, stereoisomers, and esters thereof.

2. A compound of claim 1, wherein one of W₁, W₂, W₃, W₄, and W₅ is N.

3. A compound of claim 1, wherein two of W₁, W₂, W₃, W₄, and W₅ is N.

4. A compound of claim 1, wherein R¹ is selected from the group consisting of —X¹—COR^{a}, —X¹—CO₂R^{a}, —X¹—CONR^{a}R^{b}, SO₂R^{a}, aryl, heteroaryl, substituted aryl and substituted heteroaryl.

5. A compound of claim 1, wherein D is —CH₂— or —O—.

6. A compound of claim 1, wherein E is —CH₂— or —O—.

7. A compound of claim 1, wherein D is —CH₂— and E is —O—.

8. A compound of claim 1, wherein D is —O— and E is —CH₂—.

9. A compound of claim 1 wherein Ar is selected from the group consisting of phenyl, pyridyl, pyrimidinyl, pyrazinyl, pyridazinyl, triazinyl, substituted phenyl, substituted pyridyl, substituted pyrimidinyl, substituted pyrazinyl, substituted pyridazinyl, and substituted triazinyl, and wherein when Ar is substituted, Ar is independently substituted with one or two R⁶ groups.

10. A compound of claim 9, wherein the R⁶ group is independently selected from the group consisting of halo, C₁₋₅alkyl, C₁₋₅haloalkyl, —SOR^{a}, —SO₂R^{a}, and 5-membered heteroaryl group.

11. A compound of claim 10, wherein the R⁶ group is independently selected from the group consisting of fluoro, —CH₃, —S(O)₂CH₃, N-linked tetrazolyl, N-linked triazolyl, N-linked imidazolyl, N-linked pyrazolyl and N-linked pyrrolyl.

12. A compound of claim 1, wherein zero, one or two of W₁, W₂, W₃, W₄, and W₅ is N; D and E are independently —CH₂— or —O—; Ar is selected from the group consisting of phenyl, pyridyl, pyrimidinyl, pyrazinyl, pyridazinyl, and triazinyl; R₁ is selected from the group consisting of phenyl, pyridyl, pyrimidinyl, pyrazinyl, pyridazinyl, triazinyl, substituted phenyl, substituted pyridyl, substituted pyrimidinyl, substituted pyrazinyl, substituted pyridazinyl, and substituted triazinyl, and wherein when Ar is substituted, Ar is independently substituted with one or two R⁶ groups.

13. A compound of claim 12 wherein, R⁶ is selected from the group consisting of fluoro, —CH₃, —S(O)₂CH₃, N-linked tetrazolyl, N-linked triazolyl, and N-linked imidazolyl, N-linked pyrazolyl and N-linked pyrrolyl.

14. A compound having the Formula (II) or Formula (III); 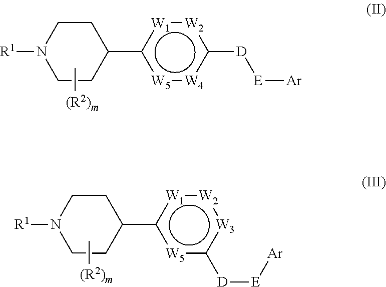
wherein:
W₁, W₂, W₃, W₄ and W₅ are independently selected from the group consisting of CR³, and N, provided that only zero, one, two, or three of W₁, W₂, W₃, W₄ and W₅ is N;
D and E are independently selected from the group consisting of a bond, —(CHR⁴)ₚ—, —C(O)—, —O—, —S—, —S(O)—, —S(O)₂—, and —NR⁵—, provided that one of D or E is —(CHR⁴)ₚ— or —C(O)— and wherein;
p is 0, 1, or 2;
m is 0, 1, 2, 3, or 4;
Ar is a 5- to 10-membered aryl or heteroaryl group, optionally substituted with from one to five R⁶ groups;
R¹ is selected from the group consisting of H, C₁₋₁₀substituted alkyl, C₃₋₇cycloalkyl, C₂₋₁₀alkenyl, C₂₋₁₀alkynyl, —X¹—COR^{a}, —X¹—CO₂R^{a}, —X¹—CONR^{a}R^{b}, —SO₂R^{a}, a 4- to 7-membered heterocyclyl group, aryl and a 5- to 10-membered heteroaryl group, wherein each of said cycloalkyl group, heterocyclyl group, aryl group and heteroaryl group is optionally substituted with from 1 to 4 substituents independently selected from the group consisting of halo, C₁₋₁₀alkyl, C₁₋₁₀substituted alkyl, C₃₋₇cycloalkyl, C₂₋₁₀alkenyl, C₂₋₁₀alkynyl, aryl, heteroaryl, —CN,
—NR^{a}COR^{b}, —NR^{a}CONR^{a}R^{b}, —NO₂, —OR^{a}, —NR^{a}R^{b}, —COR^{a}, —CO₂R^{a}, —CONR^{a}R^{b}, —SR^{a}, —S(O)R^{a}, —S(O)₂R^{a}, —NR^{a}S(O)₂R^{b}, and —SO₂NR^{a}R^{b}, or optionally R^{a} and R^{b} are combined to form a 4-, 5- or 6-membered ring, and X¹ is selected from the group consisting of a bond, C₁₋₄alkylene, C₂₋₆alkenylene, C₂₋₆alkynylene, —C(O)—, and —C(O)—(CH₂)₁₋₄—, wherein the aliphatic portions of X¹ are optionally substituted with one to three members selected from the group consisting of halo, C₁₋₄alkyl, C₁₋₄substituted alkyl and C₁₋₄haloalkyl;
each R² is independently selected from the group consisting of H, halo, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b}, and wherein when the subscript m is 2 and R² is alkyl or substituted alkyl, the two R² members can optionally cyclize to form a ring;
R³ is selected from the group consisting of H, halo, cyano, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b};
each R⁴ is independently selected from the group consisting of H, halo, C₁₋₅alkyl, C₁₋₅substituted alkyl, C₃₋₇cycloalkyl, —COR^{a}, —CO₂R^{a}, —CONR^{a}R^{b}, —OR^{a}, —NR^{a}R^{b}, —NR^{a}COR^{b}, —SOR^{a}R^{b}, —SO₂R^{a} and —SO₂NR^{a}R^{b};
R⁵ is selected from the group consisting of H, C₁₋₅alkyl, and C₁₋₅substituted alkyl;
each R⁶ is independently selected from the group consisting of H, halo, C₁₋₁₀alkyl, C₁₋₁₀substituted alkyl, C₃₋₇cycloalkyl, C₂₋₂₀alkenyl, C₂₋₁₀alkynyl, CN, NO₂, —OR^{a}, —NR^{a}R^{b}, —COR^{a}, —CO₂R^{a}, —CONR^{a}R^{b}, —NR^{a}COR^{b}, —NR^{a}CO₂R^{b}, —NR^{a}CONR^{a}R^{b}, —SR^{a}, —S(O)R^{a}, —S(O)₂R^{a}, —NR^{a}S(O)R^{b}, —NR^{a}S(O)₂R^{b}, —SO₂NR^{a}R^{b}, a 4- to 7-membered heterocyclyl group, aryl and a 5- to 10-membered heteroaryl group, wherein each of said heterocyclyl groups, said aryl and heteroaryl groups are optionally substituted with from one to four substituents independently selected from the group consisting of halo, oxo, C₁₋₄alkyl, C₁₋₄haloalkyl, C₃₋₇cycloalkyl, CN, NO₂, —OR^{a}, —NR^{a}R^{b},
—COR^{a}, —CO₂R^{a}, —CONR^{a}R^{b}, —NR^{a}COR^{b}, —NR^{a}CO₂R^{b}, —NR^{a}CONR^{a}R^{b}, —SR^{a}, —S(O)R^{a}, —S(O)₂R^{a}, —NR^{a}SO₂R^{b}, and —SO₂NR^{a}R^{b}, and optionally R^{a} and R^{b} are combined to form a 4-, 5- or 6-membered ring;
and each R^{a} and R^{b} is independently selected from the group consisting of hydrogen, C₁₋₁₀alkyl, C₁₋₁₀haloalkyl, C₃₋₁₀cycloalkyl, heterocyclyl, C₂₋₁₀alkenyl, C₂₋₁₀alkynyl, aryl, substituted aryl, 5- to 6-membered heteroaryl, 5- to 6-membered substituted heteroaryl, and arylC₁₋₄alkyl; and wherein the aliphatic portions of each of said R^{a} and R^{b} is optionally substituted with from one to three members selected from the group consisting of halo, —ORⁿ, —OCORⁿ, —OC(O)N(Rⁿ)₂, —SRⁿ, —S(O)Rⁿ, —S(O)₂Rⁿ,
—S(O)₂N(Rⁿ)₂, —NRⁿS(O)₂Rⁿ, —C(O)N(Rⁿ)₂, —C(O)Rⁿ, —NRⁿC(O)Rⁿ, —NRⁿC(O)N(Rⁿ)₂, —CO₂Rⁿ, —NRⁿCO₂Rⁿ, —CN, —NO₂, —N(Rⁿ)₂ and —NRⁿS(O)₂N(Rⁿ)₂, wherein each Rⁿ is independently hydrogen or an unsubstituted C₁₋₆alkyl;
or pharmaceutically acceptable salts, solvates, stereoisomers, and esters thereof.

15. A compound of claim 14, wherein one of W₁, W₂, W₃, W₄, and W₅ is N.

16. A compound of claim 14, wherein two of W₁, W₂, W₃, W₄, and W₅ is N.

17. A compound of claim 14, wherein R₁ is selected from the group consisting —X¹—COR^{a}, —X¹—CO₂R^{a}, —X¹—CONR^{a}R^{b}, SO₂R^{a}, aryl, heteroaryl, substituted aryl and substituted heteroaryl.

18. A compound of claim 14, wherein D is —CH₂—, or —O—.

19. A compound of claim 14, wherein E is —CH₂—, or —O—.

20. A compound of claim 14, wherein D is —CH₂— and E is —O—.

21. A compound of claim 14, wherein D is —O— and E is —CH₂—.

22. A compound of claim 14 wherein Ar is selected from the group consisting of phenyl, pyridyl, pyrimidinyl, pyrazinyl, pyridazinyl, triazinyl, substituted phenyl, substituted pyridyl, substituted pyrimidinyl, substituted pyrazinyl, substituted pyridazinyl, and substituted triazinyl, and wherein when Ar is substituted, Ar is independently substituted with one or two R⁶ groups.

23. A compound of claim 22, wherein the R⁶ group is independently selected from the group consisting of halo, C₁₋₅alkyl, C₁₋₅haloalkyl, —SOR^{a}, —SO₂R^{a}, and 5-membered heteroaryl group.

24. A compound of claim 23, wherein the R⁶ group is independently selected from the group consisting of fluoro, —CH₃, —S(O)₂CH₃, N-linked tetrazolyl, N-linked triazolyl, N-linked imidazolyl, N-linked pyrazolyl and N-linked pyrrolyl.

25. A compound of claim 14, wherein zero, one or two of W₁, W₂, W₃, W₄, and W₅ is N; D and E are independently —CH₂— or —O—; Ar is selected from the group consisting of phenyl, pyridyl, pyrimidinyl, pyrazinyl, pyridazinyl, and triazinyl; R₁ is selected from the group consisting of phenyl, pyridyl, pyrimidinyl, pyrazinyl, pyridazinyl, triazinyl, substituted phenyl, substituted pyridyl, substituted pyrimidinyl, substituted pyrazinyl, substituted pyridazinyl, and substituted triazinyl, and wherein when Ar is substituted, Ar is independently substituted with one or two R⁶ groups.

26. A compound of claim 25 wherein, R⁶ is selected from the group consisting of fluoro, —CH₃, —S(O)₂CH₃, N-linked tetrazolyl, N-linked triazolyl, and N-linked imidazolyl, N-linked pyrazol and N-linked pyrrolyl.

27. A compound of Examples 1, 2, 3, 4, 5, 6, 7, 8, 9, 10, 11, 12, 13 and 14.

28. A pharmaceutical composition comprising a pharmaceutically acceptable excipient and a compound of claim 1.

29. A method of treating a disease or condition selected from the group consisting of Type I diabetes, Type II diabetes and metabolic syndrome, said method comprising administering to a subject in need of such treatment an effective amount of a compound of claim 1.

30. The method of claim 29, wherein said disease is Type II diabetes.

31. A method of stimulating insulin production, said method comprising administering an effective amount of a compound of claim 1 to a mammal.

32. The method of claim 31, wherein said mammal is a human.

33. The method of claim 31, wherein insulin is produced by a beta cell of said mammal.

34. A method of stimulating glucose-dependent insulin secretion, said method comprising administering an effective amount of a compound of claim 1 to a mammal.

35. The method of claim 34, wherein said mammal is a human.

36. The method of claim 35, wherein insulin is produced by a beta cell of said mammal.

37. A method of lowering blood glucose in a mammal, said method comprising administering an effective amount of a compound of claim 1 to a mammal.

38. The method of claim 37 wherein said mammal is a human.

39. A method of lowering blood triglyceride levels in a mammal, said method comprising administering an effective amount of a compound of claim 1 to a mammal.

40. The method of claim 39 wherein said mammal is a human.

41-48. (canceled)
